(19) United States
(12) Patent Application Publication
(10) Pub. No.: US 2017/0189623 A1
(43) Pub. Date: Jul. 6, 2017

(54) SYRINGE AND SAFETY CAP ASSEMBLY FOR SYRINGE

(71) Applicant: UNITEKO CO., LTD., Gwangju-si, Gyeonggi-do (KR)

(72) Inventor: Hyun-Seung LEE, Guri-si, Gyeonggi-do (KR)

(73) Assignee: UNITEKO CO., LTD., Gwangju-si, Gyeonggi-do (KR)

(21) Appl. No.: 15/313,551

(22) PCT Filed: Jul. 10, 2015
(86) PCT No.: PCT/KR2015/007183

§ 371 Date: Nov. 22, 2016

(30) Foreign Application Priority Data
Jul. 21, 2014 (KR) 10-2014-0092072
Aug. 2, 2014 (KR) 10-2014-0099359
Oct. 31, 2014 (KR) 10-2014-0150770
Oct. 31, 2014 (KR) 10-2014-0150773

(51) Int. Cl. A61M 5/32; A61M 5/31

(52) U.S. Cl. CPC A61M 5/3213; A61M 5/3278; A61M 5/31; A61M 2005/3284; A61M 2005/3215; A61M 2205/7545; A61M 2205/0216; A61M 2209/084

(57) ABSTRACT

The present invention relates to a syringe and a syringe safety cap assembly and, specifically, to a syringe safety cap assembly capable of preventing a user from being injured when a syringe needle is inserted into a cap and completely preventing reuse of the syringe or the syringe needle to be discarded. In addition, the present invention relates to the syringe having a valve member capable of filtering foreign substances during the inflow of a drug and restraining the filtered foreign substances from being discharged during the discharge of the drug, when foreign substances such as glass dust are mixed with substances such as a syringe drug. The present invention provides the syringe safety cap assembly comprising: an accommodation cap having a space into which at least a part of the syringe needle and a syringe body can be inserted and accommodated, and of which the shape is deformed so as to cause the shape of the syringe needle or the syringe body accommodated therein to be deformed when external force is applied; and a support part connected to the accommodation cap, thereby providing support power so as not to fall down when the accommodation cap is placed on the floor, wherein the support part has a curved outline, thereby guiding the accommodation cap so as to be ultimately erected on the floor while guiding the accommodation cap so as to swing left and right when placed on the floor.

### TECHNICAL FIELD

The present invention relates to a syringe and a safety cap assembly for a syringe, and more particularly, to a safety cap assembly for a syringe, which is capable of preventing a user from being injured when inserting a syringe needle into a cap and of fundamentally preventing the reuse of syringes or syringe needles that are to be discarded.

Further, the present invention relates to a syringe having a valve member, which is capable of filtering foreign substances such as fine glass splinters, if any, from a liquid medication while the liquid medication is introduced into the syringe and of preventing the filtered foreign substances from being discharged while the liquid medication is discharged.

### BACKGROUND ART

There are frequent cases in which, after a general disposal syringe is used, a user is pricked by a needle while inserting the needle into a needle storage cap and is infected with pathogenic bacteria, and in which innocent patients catch diseases due to the reuse of needles, which should be prevented.

In order to prevent a syringe user from being unexpectedly injured or infected with a disease, safety syringes of various types have been devised, representative examples of which are disclosed in Korean Patent Publication No. 10-2003-0043209 and Korean Patent Publication No. 2001-0040365.

However, such safety syringes have shortcomings in that they are comparatively expensive and a user must use both of his/her hands in order to handle a needle.

Further, there are no known methods or devices for enabling a user to easily realize both a reusable mode and an unreusable mode of a syringe using a safety cap assembly.

On the other hand, as disclosed in Korean Patent Publication No. 10-2011-137560, a syringe generally comprises a cylindrical-shaped cylinder having a space for accommodating an injection liquid therein, a needle-mounting part formed at the front end portion of the cylinder, and a needle mounted to the needle-mounting part.

The syringe further comprises a plunger, which is inserted into the cylinder so as to move up and down in order to suck an injection liquid into the cylinder or to discharge the injection liquid out of the cylinder.

In order to inject an injection liquid into a patient using such a conventional syringe, the plunger is pushed to closely contact the inner front end surface of the cylinder, a glass ampoule filled with an injection liquid is opened by snapping off the neck of the glass ampoule, the syringe needle is dipped into the injection liquid in the glass ampoule, and the plunger is pulled back to suck the injection liquid into the cylinder.

However, fine glass splinters, which are formed when the glass ampoule is opened by snapping off the neck of the glass ampoule, are mixed with the injection liquid in the glass ampoule.

The fine glass splinters mixed with the injection liquid are also introduced into the cylinder when the injection liquid is sucked into the syringe, and the fine glass splinters injected into a human body with the injection liquid can cause death or serious injury to the patient.

### DISCLOSURE

### Technical Problem

The present invention has been made to solve the above problems with the prior art, and it is an object of the present invention to provide a safety cap assembly for a syringe, which is capable of maintaining a safety cap in an upright orientation on a horizontal surface utilizing the principle of a roly-poly toy and of enabling a user to easily insert a syringe needle into the safety cap using only one hand, which is holding the syringe.

It is another object of the present invention to provide a safety cap assembly for a syringe, which is capable of allowing or fundamentally preventing the reuse of a syringe depending on whether the syringe is intended to be reused and on the pressure with which the syringe is inserted into the safety cap.

It is a further object of the present invention to provide a safety cap assembly for a syringe, which is capable of fundamentally preventing the reuse of a syringe needle or a syringe that is to be discarded by enabling a user to apply external force to a cap, in which a main body of a syringe or a syringe needle is inserted, so that the main body of the syringe or the syringe needle is deformed or damaged in the state of being securely accommodated in the cap.

It is another further object of the present invention to provide a syringe, which is capable of preventing foreign substances such as fine glass splinters from being injected into a human body due to a valve member, which performs different operations depending on the processes of sucking a liquid into a syringe cylinder and of discharging the liquid from the syringe cylinder using a syringe plunger.

It is yet another further object of the present invention to provide a syringe, which includes a valve member that is configured as a diaphragm capable of being simply operated depending on a change in the pressure gradient in the anterior-posterior direction, rather than a valve member having a plurality of components and complicated coupling relationships therebetween.

### Technical Solution

In accordance with an aspect of the present invention, the above and other objects can be accomplished by the provision of a safety cap assembly for a syringe including an inner cap for receiving a needle and a needle-mounting part of the syringe inserted thereinto, an outer cap for accommodating the inner cap therein, the outer cap having a deformation guide slot formed in a side surface thereof so as to be spread by a predetermined pressure applied thereto, a connection part for connecting the inner cap and the outer cap, the connection part being configured so as to be elastically deformed, and a support part connected to at least one of the connection part and the outer cap, the support part supporting and maintaining the inner cap, the outer cap and the connection part spaced apart from a horizontal surface and providing a support force to prevent the inner cap, the outer cap and the connection part from falling down.

In accordance with another aspect of the present invention, a safety cap assembly for a syringe includes an accommodation cap having a space formed therein to accommodate a needle and at least a part of a main body of the syringe inserted thereinto, the accommodation cap being configured so as to be deformed by an external force applied thereto, thereby causing deformation of the needle or the main body of the syringe accommodated therein, and a support part connected to the accommodation cap, the support part providing a support force so as to prevent the accommodation cap placed on a horizontal surface from falling down.

In accordance with a further aspect of the present invention, a safety cap assembly for a syringe includes an accommodation cap having a space formed therein to accommodate a needle and at least a part of a main body of the syringe inserted thereinto, the accommodation cap being configured so as to be deformed by an external force applied thereto, thereby causing deformation of the needle or the main body of the syringe accommodated therein, wherein, when the accommodation cap is deformed while the needle or at least a part of the main body of the syringe is accommodated in the accommodation cap, the accommodation cap forms an escape prevention structure for preventing the deformed needle or a deformed or broken portion of the main body of the syringe from escaping outside the accommodation cap.

In accordance with another further aspect of the present invention, a syringe includes a needle-mounting part for supporting a needle, a filter member provided in the needle-mounting part and mounted to an end of the needle, a filter cover disposed outside the filter member and having a through-hole formed in an outer peripheral surface thereof and an opening formed in an end portion thereof, and a valve member disposed on an outer peripheral surface of the filter cover, wherein, when a liquid is introduced into the syringe by an introduction operation of a plunger, the valve member segregates a space in the needle-mounting part so as to induce introduction of the liquid toward the opening, and when the liquid is discharged by a discharge operation of the plunger, the valve member releases the segregation of the space in the needle-mounting part so as to induce discharge of the liquid via the through-hole and forms a passage for discharge of the liquid, the passage for discharge of the liquid being different from a passage for introduction of the liquid.

### Advantageous Effects

According to the above-described present invention, since a syringe user is able to cover a needle with a cap using only the hand that is holding a syringe after using the syringe, there is no possibility of the other hand being pricked by the needle, thereby ensuring safety for the user.

Further, a syringe needle is bent, or a head portion of a main body of a syringe is broken, merely by inserting a syringe needle, a needle-mounting part, and a main body of a syringe, to which the needle-mounting part is coupled, into a safety cap assembly, and by bending an accommodation cap, thereby making it completely impossible to reuse the syringe.

Therefore, there is an advantage in that the intended or unintended reuse of a main body of a syringe or a syringe needle is completely prevented.

As a result, it is possible to prevent infection attributable to the reuse of a syringe.

According to the present invention, since a safety cap is always oriented upwards utilizing the principle of a roly-poly toy, it is easy to insert a needle and a needle-mounting part into the safety cap.

Further, due to a support part for supporting a safety cap assembly so as to be maintained in an upright orientation, even when a syringe user carelessly places the safety cap assembly on a horizontal surface, it naturally rights itself without the necessity for the user to carefully stand the safety cap assembly upright, thereby increasing convenience in use.

On the other hand, when it is necessary to reuse a syringe needle, for example, when a syringe is to be repeatedly used only for one specific patient, a user may insert a needle and a needle-mounting part into a safety cap with a small force and then may withdraw them back out in order to reuse them.

Meanwhile, when a user wants to prevent other people from being infected with pathogenic bacteria by fundamentally preventing the reuse of a syringe needle, he/she presses the syringe down into the safety cap assembly with a predetermined pressure or higher so that a needle-mounting part moves downwards and is caught by a support ring or a support protrusion, which prohibits the upward movement of the needle-mounting part and makes it very difficult to withdraw the needle and the needle-mounting part back out.

Therefore, a user may easily realize a mode of reusing a needle and a needle-mounting part or a mode of discarding them depending on his/her selection.

In addition, a syringe according to the present invention has an advantage in that, when foreign substances such as fine glass splinters are mixed with a liquid medication in a glass ampoule and the liquid medication is sucked into the syringe, the foreign substances are filtered out by a filter member and thus are prevented from entering a syringe cylinder.

At this time, since a valve member is maintained in a closed state, the liquid does not flow through a relatively short passage, directed toward through-holes, in the filter member, whereby foreign substances do not move toward the through-holes, and are filtered out by the filter member while moving toward an opening.

Meanwhile, when the liquid in a syringe cylinder is discharged outside, the valve is opened by a liquid discharge pressure, and a bypass passage is created around the filter cover, whereby most of the liquid in the syringe cylinder is discharged through the needle via the bypass passage, at which time, since the liquid that is discharged applies a force to foreign substances in the direction opposite the discharge direction, the foreign substances remain in the filter member.

Therefore, there is an advantage of preventing foreign substances from entering a human body when a liquid medication is injected into the human body using a syringe.

On the other hand, since a valve member is configured as a diaphragm having a structure capable of allowing the flow of liquid in one direction and prohibiting the flow of liquid in the opposite direction, rather than a complicated structure, there is also an advantage in that the structure becomes simpler in the limited inner space of the needle-mounting part.

### DESCRIPTION OF DRAWINGS

The above and other objects, features and other advantages of the present invention will be more clearly understood from the following detailed description taken in conjunction with the accompanying drawings, in which:

FIG. 1 is a perspective view illustrating the state in which a needle and a needle-mounting part are inserted into a safety cap assembly for a syringe according to a first embodiment of the present invention;

FIG. 2 is a partially perspective view illustrating the state in which a needle and a needle-mounting part are inserted into the safety cap assembly for a syringe according to the first embodiment of the present invention;

FIG. 3 is a partially perspective view of the safety cap assembly for a syringe according to the first embodiment of the present invention;

FIG. 4 is a perspective view illustrating a locking part and a support ring surrounding an inner cap in the safety cap assembly for a syringe according to the first embodiment of the present invention;

FIG. 5 is a perspective view of the inner cap that constitutes the safety cap assembly for a syringe according to the first embodiment of the present invention;

FIG. 6 is a view illustrating the state before the needle and the needle-mounting part are inserted into the safety cap assembly for a syringe according to the first embodiment of the present invention;

FIG. 7 is a perspective view illustrating the state in which the needle and the needle-mounting part are inserted into the safety cap assembly for a syringe according to the first embodiment of the present invention in a reusable mode;

FIG. 8 is a sectional view illustrating the state in which the needle and the needle-mounting part are inserted into the safety cap assembly for a syringe according to the first embodiment of the present invention in the reusable mode;

FIGS. 9 and 10 are internal perspective views illustrating the state in which the needle and the needle-mounting part are inserted into the safety cap assembly for a syringe according to the first embodiment of the present invention in an unreusable mode;

FIG. 11 is a perspective view illustrating the state in which the needle and the needle-mounting part are inserted into a safety cap assembly for a syringe according to a second embodiment of the present invention;

FIG. 12 is a partially perspective view illustrating the state in which the needle and the needle-mounting part are inserted into the safety cap assembly for a syringe according to the second embodiment of the present invention in a reusable mode;

FIG. 13 is a plan view illustrating the top surface of the safety cap assembly for a syringe according to the second embodiment of the present invention;

FIGS. 14 and 15 are partially perspective views illustrating the state before the needle and the needle-mounting part are inserted into the safety cap assembly for a syringe according to the second embodiment of the present invention;

FIGS. 16 and 17 are internal perspective views illustrating the state in which the needle and the needle-mounting part are inserted into the safety cap assembly for a syringe according to the second embodiment of the present invention in an unreusable mode;

FIG. 18 is a view illustrating the state before the needle and the main body of the syringe are inserted into a safety cap assembly according to a third embodiment of the present invention;

FIG. 19 is a view illustrating the state after the needle and the main body of the syringe are inserted into the safety cap assembly according to the third embodiment of the present invention;

FIGS. 20 and 21 are views illustrating the detailed configuration of the safety cap assembly according to the third embodiment of the present invention;

FIG. 22 is a view illustrating the state in which a deformation prevention part is separated from an accommodation cap in the safety cap assembly according to the third embodiment of the present invention;

FIGS. 23 to 27 are views illustrating the state in which the needle and the main body of the syringe are deformed or broken when the accommodation cap is deformed in the safety cap assembly according to the third embodiment of the present invention;

FIG. 28 is a view illustrating the main body of a syringe that has been broken;

FIG. 29 is a perspective view illustrating the appearance of a syringe according to the present invention;

FIG. 30 is a side view illustrating a filter mounted to the needle of the syringe according to the present invention;

FIG. 31 is a side view illustrating a filter cover mounted to the needle of the syringe according to the present invention;

FIG. 32 is a side view illustrating a valve member and a guide member mounted to the syringe according to the present invention;

FIG. 33 is a side-sectional view illustrating the valve member and the guide member mounted to the syringe according to the present invention;

FIG. 34 is a rear perspective view of the valve member and the filter cover in the syringe according to the present invention;

FIG. 35 is a front perspective view of the valve member and the filter cover in the syringe according to the present invention;

FIG. 36 is a side-sectional view illustrating the state in which a liquid is introduced into the syringe according to the present invention; and

FIG. 37 is a side-sectional view illustrating the state in which the liquid is discharged from the syringe according to the present invention.

### BEST MODEL

Advantages and features of the present invention and methods for achieving them will be made clear from the embodiments described below in detail with reference to the accompanying drawings.

The present invention may, however, be embodied in many different forms and should not be construed as being limited to the embodiments set forth herein. Rather, these embodiments are provided so that this disclosure will be thorough and complete, and will fully convey the scope of the invention to those skilled in the art. The present invention is merely defined by the scope of the claims.

Also, terms used in the following description are used only to describe the embodiments and are not intended to restrict the present invention.

In the specification, the expression of singularity includes a plural meaning unless the singular expression is explicitly different in context. It will be further understood that the terms “comprises” and/or “comprising,” when used in this specification, specify the presence of stated components, but do not preclude the presence or addition of other components.

Unless otherwise defined, all terms used herein (including technical and scientific terms) have the same meaning as commonly understood by those skilled in the art to which this invention pertains.

Hereinafter, a first embodiment of the present invention will be described with reference to the annexed drawings.

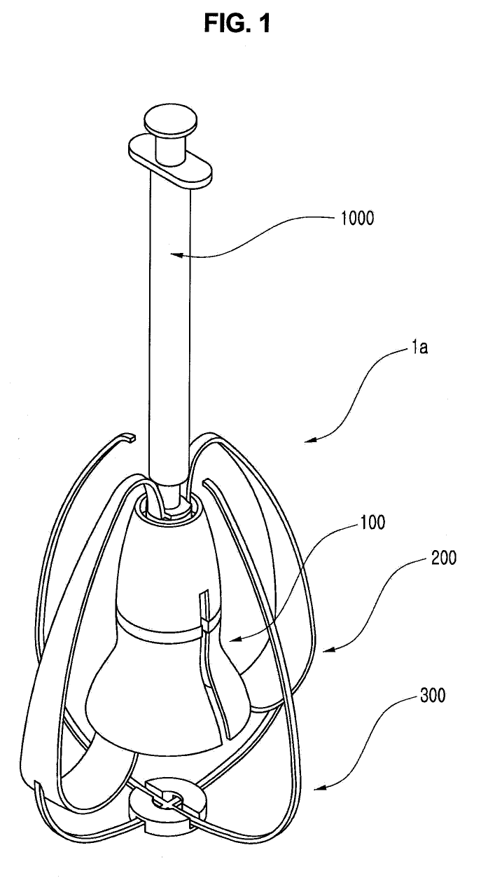

As illustrated in FIGS. 1 to 3 and 6, a syringe safety cap assembly 1a (hereinafter, referred to as a “safety cap assembly”) according to the first embodiment of the present invention may receive a needle 1200 and a needle-mounting part 1100 of a syringe 1000, which are inserted into the top portion of the safety cap assembly, and may stably stand upright on a horizontal surface due to a support part 300, which will be described later.

The safety cap assembly 1a comprises a cap 100, into which the needle 1200 and the needle-mounting part 1100 are inserted, and the cap 100 includes a cylindrical-shaped inner cap 110 and an outer cap 120 in which the inner cap 110 is accommodated.

The inner cap 110 may be formed to have a cylindrical shape, whereas the outer cap 120 may be formed such that the diameter of the lower end portion thereof is larger than the diameter of the upper end portion thereof, which is for the purpose of facilitating connection with a connection part 200, which will be described later, and of facilitating deformation.

The inner cap 110 and the outer cap 120 are connected to each other by the connection part 200.

The connection part 200 may be preferably formed of an elastic material.

The connection part 200 connects the upper end portion of the inner cap 110 and the lower end portion or the side surface of the outer cap 120.

To this end, the connection part 200 includes a first connection portion 210, which is connected to the upper end portion of the inner cap 110, a second connection portion 220, which is connected to the lower end portion or the side surface of the outer cap 120, and a third connection portion 230, which connects the first connection portion 210 and the second connection portion 220.

The third connection portion 230 is disposed outside the outer cap 120. It is preferable that the connection part 200 be provided in a pair and that the pair of connection parts 200 be arranged so as to face each other while sandwiching the outer cap 120 therebetween; however, the embodiment is not limited thereto.

The connection part 200 may be preferably formed as a ring-shaped handle.

Meanwhile, it is preferable for the lower end of the connection part 200 to be located below the lower end of the outer cap 120, which is for the purpose of enabling the lower end of the outer cap 120 to spread due to deformation of the lower end of the connection part 200, which occurs when the lower end of the connection part 200 is brought into contact with a horizontal surface.

To this end, the outer cap 120 has a deformation guide slot 121 formed therein.

It is preferable that the deformation guide slot 121 be provided in a pair and that the pair of deformation guide slots 121, each of which is located between the connection parts 200, be arranged so as to face each other.

It is preferable for the deformation guide slot 121 to extend vertically along the outer peripheral surface of the outer cap 120 and for the lower end of the deformation guide slot 121 to be open.

Therefore, when the respective connection parts 200 are brought into contact with a horizontal surface and are thus deformed, the connection parts 200 pull the lower end portion of the outer cap 200 in opposite directions.

When the lower end portion of the outer cap 200 is pulled in opposite directions, the lower portion of the outer cap 120 may be easily spread by the deformation guide slots 121.

The reason why the lower portion of the outer cap 120 must be spread will be described later.

In order to enable a user to insert the needle 1200 and the needle-mounting part 1100 into the cap 100 using only the hand that is holding the syringe 1000, the insertion hole in the cap 100 must be oriented upwards. That is, the cap 100 must be maintained in the state of standing upright.

To this end, the safety cap assembly 1a according to the present invention has a structure that utilizes the principle of a roly-poly toy so that it cannot be pushed over when placed on a horizontal surface.

The safety cap assembly 1a according to the present invention is supported by the support part 300 so as to be stably placed on a horizontal surface.

The support part 300 includes a support plate 330, which is configured to contact a horizontal surface and has a predetermined contact area, a first support portion 310, which passes through the support plate 330 in a first direction and has two ends connected to the connection part 200, and a second support portion 320, which passes through the support plate 330 in a second direction so as to cross the first support portion 310 and has two ends bent upwards.

Each of the first and second support portions 310 and 320 may preferably have a substantially U-shape in section.

Although the support plate 330 is illustrated as being embodied as a circular plate or a circular ring, the support plate 330 may have other shapes.

Meanwhile, the first and second support portions 310 and 320 may be preferably embodied as an elastic wire of a plastic material; however, they may also be embodied as a plate having a given width, without being limited to the configuration of a wire, and the material thereof is not limited to plastic, as long as it has elasticity.

The inner cap 110, the outer cap 120 and the connection part 200 are disposed so as to be spaced apart from a horizontal surface, that is, are kept away from the horizontal surface by the support part 300.

In order to ensure that the inner cap 110 and the outer cap 120 stand upright utilizing the principle of a roly-poly toy, the support plate 330 is located directly below the outer cap 120 so as to serve as a center support point, and the first support portion 310 extends from the support plate 301 in opposite directions so as to be symmetric to each other like a decalcomania.

This is identically applied to the second support portion 320.

Accordingly, when the support plate 301 is placed on a horizontal surface, the first support portion 310 extends symmetrically in opposite directions and the second support portion 320 also extends symmetrically in opposite directions, thereby enabling the safety cap assembly 1 to stand stably.

Further, since the first and second support portions 310 and 320 are all bent in the shape of a curved line, even when the safety cap assembly is not placed upright on a horizontal surface, it wobbles like a roly-poly toy and finally stands upright.

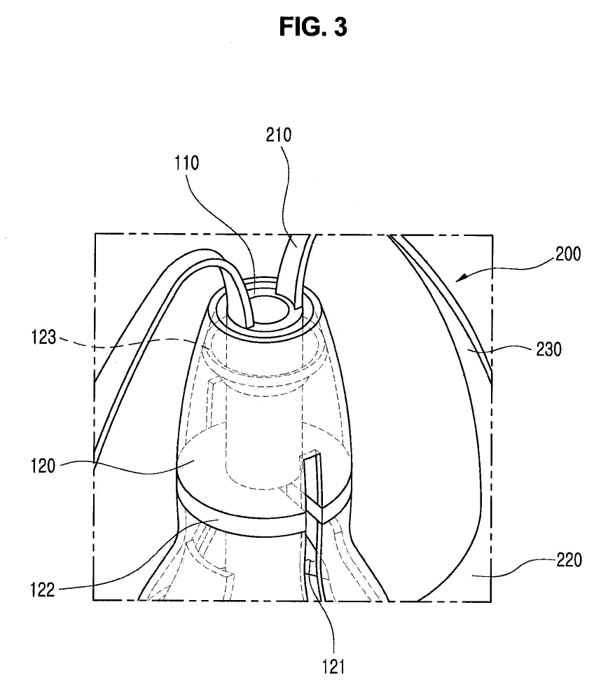

As illustrated in FIGS. 3 to 5, a locking part 122 and a support ring 123 are provided between the outer cap 110 and the inner cap 120, which is accommodated in the outer cap 110.

The locking part 122 is coupled to the outer cap 120, and is preferably embodied as a protrusion that protrudes a given length inwards from the inner surface of the outer cap 120.

The locking part 122 is provided in a pair, and the pair of locking parts 122 extend along the inner peripheral surface of the outer cap 120 and are kept separate from each other by the deformation guide slots 121, which serve as a boundary between the locking parts 122.

That is, the locking parts 122 may have a roughly ring-shaped design having cut portions formed at positions corresponding to the deformation guide slots 121.

Owing to this structure, when the outer cap 120 is spread, the locking parts 122 may also be spread away from each other.

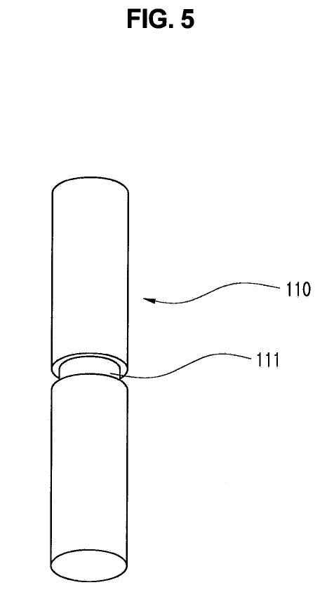

On the other hand, the inner cap 110 has a locking recess 111 formed in the outer peripheral surface thereof, in which the locking parts 122 are fitted and locked (refer to FIG. 5).

Therefore, before the outer cap 120 is spread, the locking parts 122 are locked in the locking recess 111, thereby preventing the inner cap 110 from moving downwards.

However, when the outer cap 120 and the locking parts 122 are spread together, the locking parts 122 are separated from the locking recess 111, thereby enabling the inner cap 110 to move downwards when the inner cap 110 is pressed from above.

The support ring 123 is disposed above the locking parts 122.

The support ring 123 protrudes inwards from the inner surface of the outer cap 120 so as to be in contact with the outer peripheral surface of the inner cap 110 and to surround the inner cap 110.

When the syringe 1000 is in a state of not being inserted into the cap 100, or the needle 1200 and the needle-mounting part 1100 of the syringe 1000 are inserted into the inner cap 110 with a pressure lower than a predetermined value, the upper end of the inner cap 110 is located above the support ring 123.

However, if the needle 1200 and the needle-mounting part 1100 are pressed with a predetermined pressure or higher in the state of being inserted into the inner cap 110, which will be described later, the support part 300 and the connection part 200 are deformed, the lower end portion of the outer cap 120 is spread, the locking parts 122 are separated from the locking recess 110, and consequently the inner cap 110 moves downwards.

At this time, the upper end of the inner cap 110 may be located below the support ring 123.

The support ring 123 has a sectional shape such that the inner side surface thereof extends downwards and is slanted inwards so as to guide the smooth downward movement of the inner cap 110.

However, when the upper end of the inner cap 110 is located below the support ring 123, the support ring 123 shrinks to a smaller size than when the support ring 123 is in contact with the outer peripheral surface of the inner cap 110 to support the inner cap 110, thereby stopping the upper end of the inner cap 110 and preventing the inner cap 110 and the needle-mounting part 1100 fitted to the inner cap 110 from moving upwards.

Since the needle-mounting part 1100 is kept from moving upwards and being separated from the cap, reuse of the syringe, specifically, reuse of the needle 1200, may be fundamentally prevented.

Hereinafter, the operation of the first embodiment of the present invention will be described.

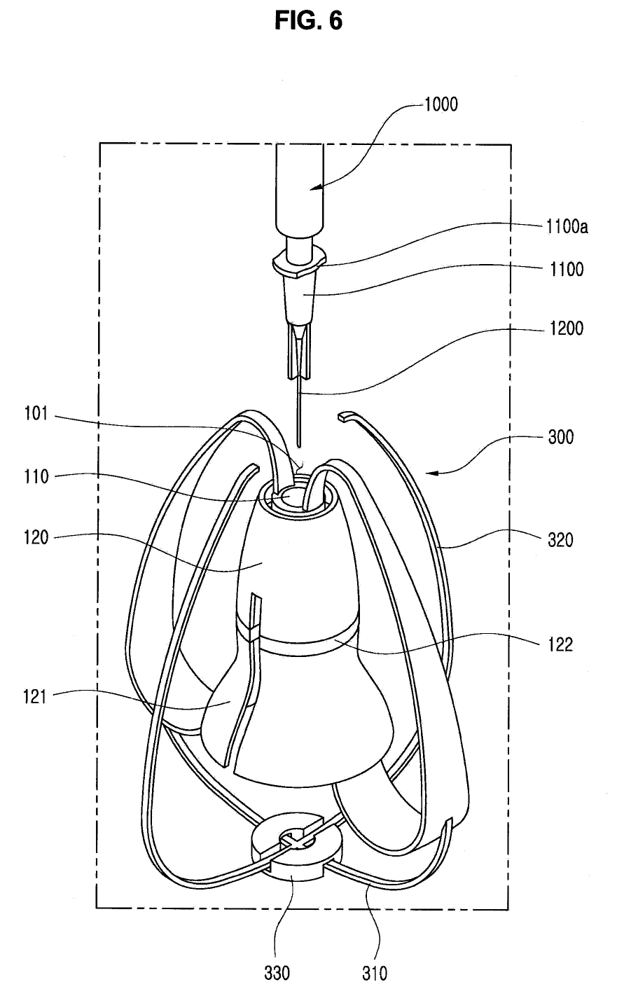

As illustrated in FIG. 6, when a user places the safety cap assembly 1a according to the first embodiment of the present invention on a horizontal surface, the safety cap assembly 1a stands upright due to the support part 300 and an insertion hole 101 in the cap 100 is directed upwards.

In this state, the user holds the syringe 1000, to which the needle 1200 and the needle-mounting part 1100 are coupled, with one hand, and inserts the needle 1200 and the needle-mounting part 1100 into the insertion hole 101 in the cap 100.

When the user inserts the needle 1200 and the needle-mounting part 1100 into the cap 100, the needle 1200 and the needle-mounting part 1100 can move until a stopping protrusion 120 formed at the upper end of the needle-mounting part 1100 contacts the upper end of the inner cap 120.

At this time, the outer cap 120 and the inner cap 110 may move downwards a small distance in the direction of the arrow in FIG. 1, and the connection part 200 and the support part 300 may be elastically deformed to a certain extent, but this is restorable elastic deformation.

This is for the purpose of reuse of the syringe 1000 (reuse only for one specific patient), and the syringe 1000 may be withdrawn back out of the cap 100 from the inserted state.

This will be referred to as a reusable mode.

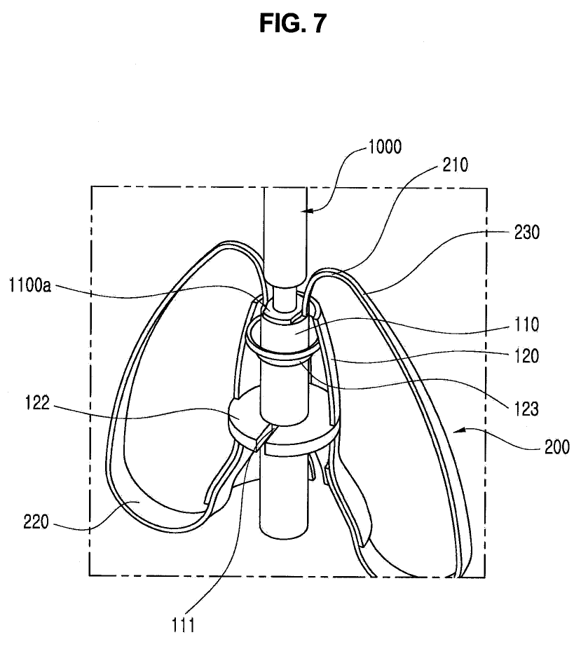

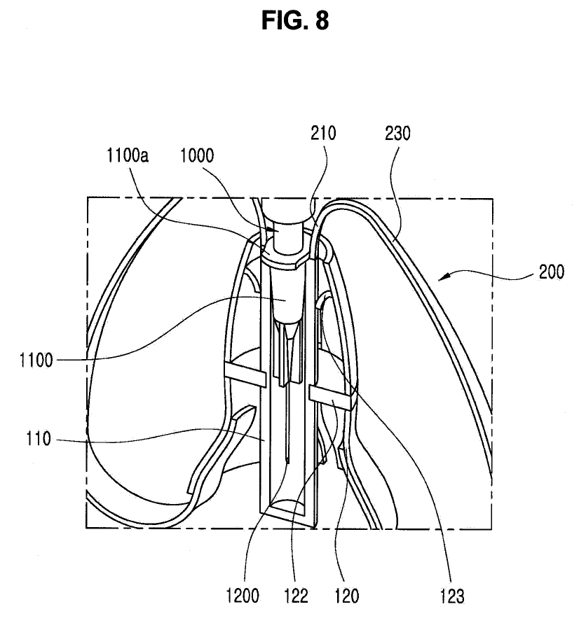

In the reusable mode, as illustrated in FIGS. 7 and 8, since the locking parts 122 formed at the outer cap 120 are locked in the locking recess 111 in the inner cap 110, the inner cap 110 is prevented from moving downwards.

On the other hand, the support ring 123 is in contact with the outer peripheral surface of the inner cap 110 and surrounds the same. The upper end of the inner cap 110 is located above the support ring 123, and in this state the support ring 123 does not play any particular role.

If the user strongly presses the syringe down in this state in order to prevent reuse of the needle 1200, the inner cap 110 and the outer cap 120 move further downwards than in the reusable mode, and the connection part 200 is deformed and the lower end thereof comes into contact with a horizontal surface.

This will be referred to as an unreusable mode.

In the unreusable mode, if the user continues to press the syringe down in the state in which the connection part 200 is in contact with a horizontal surface, the connection part 200 is deformed in the outward direction, which creates force that pulls the lower end of the outer cap 120 in the outward direction.

Due to the deformation guide slots 121 formed in the outer cap 120, two opposite portions of the lower end of the outer cap 120 move in opposite directions with respect to the deformation guide slots 121.

Accordingly, the two locking parts 122 formed at the outer cap 120 are spread and are separated from the locking recess.

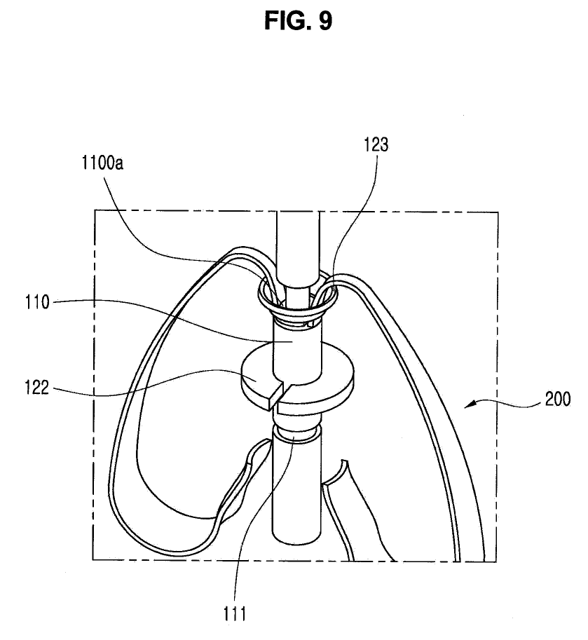

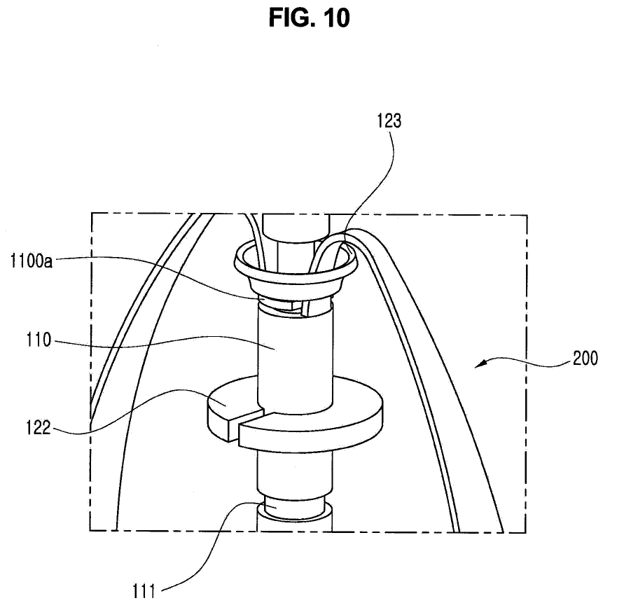

As illustrated in FIGS. 9 and 10, when the locking parts 122, which served as a stopper for keeping the inner cap 110 at its original position, are separated from the locking recess 111, the inner cap 110 moves downwards due to the downward pressure applied by the user.

At this time, the locking parts 122 are located above the locking recess 110.

Meanwhile, when the inner cap 110 moves downwards, the upper end of the inner cap 110 and the stopping protrusion 1100a of the needle-mounting part 1100, which is placed on the upper end of the inner cap 110, pass through the support ring 123 and are located below the support ring 123.

Since the support ring 123 has a sectional shape such that the inner side surface thereof extends downwards and is slanted inwards, the inner cap 110 and the stopping protrusion 1100a of the needle-mounting part 1100 may easily pass through the support ring 123.

However, after the upper end of the inner cap 110 and the stopping protrusion 1100a of the needle-mounting part 1100 pass through the support ring 123, the hole formed in the middle of the support ring 123 shrinks due to its elastic force.

Accordingly, the inner periphery of the support ring 123 covers the top surface of the stopping protrusion 1100a of the needle-mounting part 1100.

This means that the inner cap 110 and the needle-mounting part 1100 are prevented from moving upwards.

Due to this movement prevention structure, the user cannot withdraw the needle 1200 or the needle-mounting part 1100 out of the cap 100, and accordingly, it is impossible to reuse the needle 1200 or the needle-mounting part 1100.

In order to realize this unreusable mode, the user must press the syringe 1000 down with a predetermined pressure or higher so that the connection part 200 and the support part 300 are respectively spread in the opposite lateral directions.

Particularly, it is preferable for the user to press the syringe down to such an extent that the connection between the support portions of the support part 300 and the support plate 330 is cut.

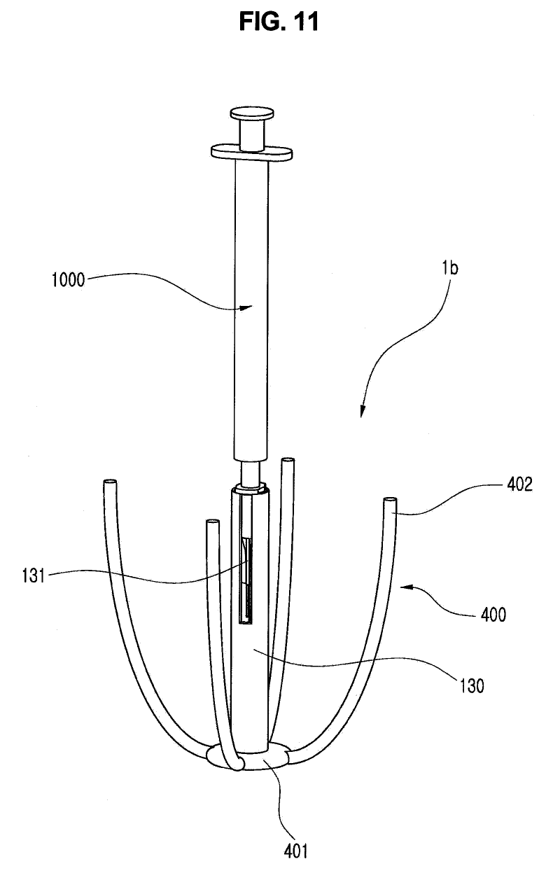

FIG. 11 is a view illustrating a second embodiment of the present invention.

A safety cap assembly 1b according to the second embodiment of the present invention does not have the double-cap structure of the first embodiment, but has a single-cap structure.

However, like the first embodiment, an insertion hole in a cap 130 is kept directed upwards utilizing the principle of a roly-poly toy.

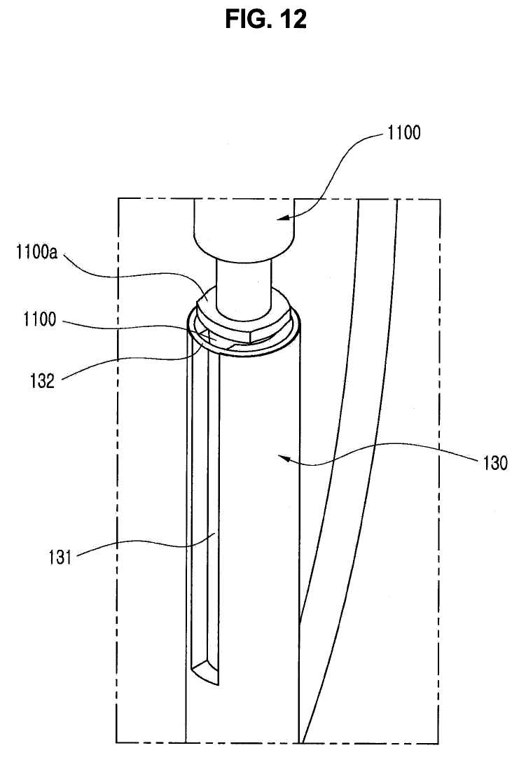

As illustrated in FIGS. 11 and 12, the safety cap 1b according to the second embodiment of the present invention comprises a cap 130, which has a cylindrical shape, and a support part 400, which is connected to the lower end portion of the cap 130 so as to keep the cap 130 in the state of standing upright.

The cap 130 may have a deformation guide portion 131 formed therein, and the deformation guide portion 131 may be embodied as a slot that extends in the vertical direction (or in the longitudinal direction of the cap) on the outer peripheral surface of the cap 130.

The deformation guide portion 131, as illustrated in FIGS. 11 and 12, may be embodied as a slot that is cut through the side wall of the cap 130; alternatively, the deformation guide portion 131 may be embodied as a membrane that is formed by making a portion of the side wall of the cap 130 thinner than the remaining portion, the membrane being torn when a predetermined pressure or higher is applied thereto, thereby spreading the cap 130.

In the case in which the deformation guide portion 131 is embodied as a slot, the upper end thereof is open, which functions to facilitate the spread of the upper end portion of the cap 130.

Further, a connection member 132, which has a ring shape or a closed-loop shape, is provided on the circumference of the upper end of the cap 130.

The connection member 132 may be embodied as a wire, which is made of resin, metal, an elastic material, or another material, as long as it breaks when subjected to a predetermined pressure or higher.

The connection member 132 is designed to endure a pressure lower than a predetermined value so as to prevent the deformation guide portion 131 from being spread, and is designed to break under a predetermined pressure or higher so as to enable the deformation guide portion 132 to be spread.

That is, when the pressure applied to the cap 130, that is, the syringe insertion pressure, is lower than a predetermined value, the connection member 132 serves as a member for maintaining the shape of the cap 130 by preventing the cap 130 from being deformed or spread.

Alternatively, as described above, in the case in which the deformation guide portion 131 is not embodied as a slot but is embodied as a membrane that is thinner than the side wall of the cap, the relatively thin membrane serves as a member for maintaining the shape of the cap 130 when subjected to a pressure lower than a predetermined value, without the connection member 132.

The connection member 132 or the shape-maintaining member may also be provided on the cap 130 or a stopping part 134, which will be described later.

Meanwhile, the support part 400 includes a support plate 401, which is disposed at the lower end portion of the cap, and a support portion 402, which extends from the support plate 401 in the radial direction of the support plate 401.

The support portion 402 may have a distal end portion that is bent upwards, and preferably, the support portion 402 may be provided in a plural number and the plurality of support portions 402 may extend in different directions. In this embodiment, four support portions 402 are provided, and each pair of support portions 402 has a U-shape in section about the support plate 401.

Accordingly, even when the safety cap assembly 2 is placed on a horizontal surface at an orientation other than upright, it wobbles naturally like a roly-poly toy and finally stands upright.

The support plate 401 serves as a center of mass, and the support portions 402, which are bent in the shape of a curved line, perform the wobbling movement, thereby enabling the safety cap assembly to finally stand upright.

The support portions 402 may be preferably embodied as an elastic wire of a plastic material; however, any material may be used, without limitation to plastic, as long as it has elasticity.

This is for the purpose of being folded when stored for packaging the product.

Like the first embodiment, the second embodiment of the present invention is capable of realizing both the reusable mode and the unreusable mode.

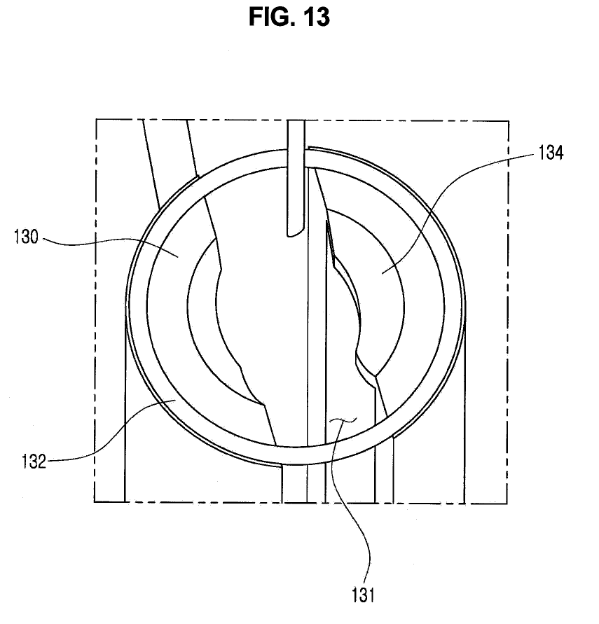

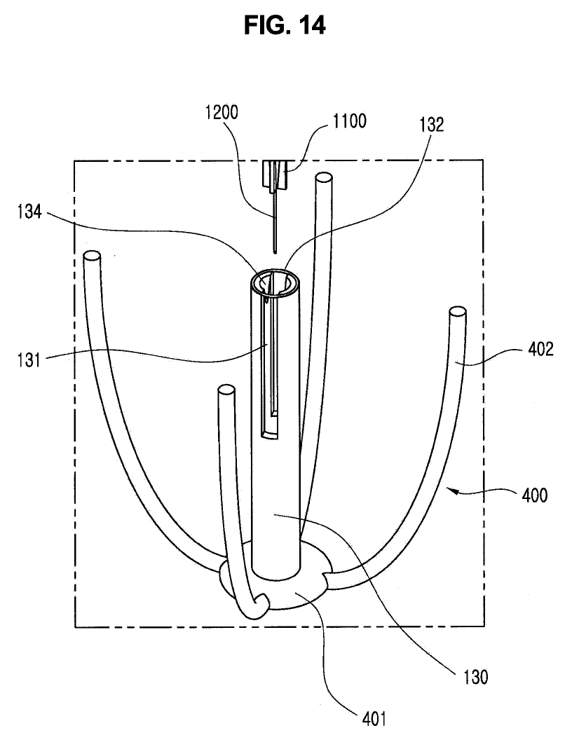

To this end, as illustrated in FIGS. 13 and 14, a stopping part 134 is provided in the cap 130.

The stopping part 134 is disposed adjacent to the inlet hole in the cap 130, and has a sectional shape such that the inner side surface thereof extends downwards and is slanted inwards.

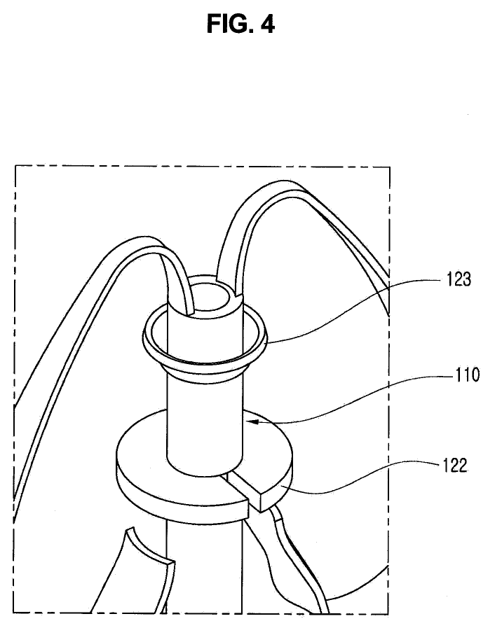

This shape is similar to or the same as the shape of the support ring 123 (refer to FIG. 4) in the first embodiment.

The stopping part 134 is provided in a plural number and the plurality of stopping parts are spaced apart from each other. Therefore, the stopping parts, which will be described later, are temporarily spread away from each other in the unreusable mode and cover the stopping protrusion 1100a of the needle-mounting part 1100.

When the user inserts the needle 1200 and the needle-mounting part 1100 into the cap 130 such that the stopping protrusion 1100a of the needle-mounting part 1100 is lightly placed on the stopping parts 134, the needle 1200 and the needle-mounting part 1100 are capable of being reused later.

However, in this reusable state, if the user presses the syringe 1000 down more strongly than for the reusable mode, the connection member 133 breaks, the deformation guide portion 131 of the slot type is widened, and the upper end portion of the cap 130 is spread.

Accordingly, the stopping parts 134 are spread away from each other, the needle-mounting part 1100 moves downwards into the gap between the stopping parts 134, which are spread, and the stopping protrusion 1100a of the needle-mounting part 1100 is located below the stopping parts 134.

Subsequently, the upper end portion of the cap, in which the stopping parts 134 are disposed, retracts back due to its elastic restoring force, and the stopping parts 134 cover the stopping protrusion 1100a formed at the upper end of the needle-mounting part 1100, thereby preventing the needle 1200 and the needle-mounting part 1100 from moving upwards.

On the other hand, in the case in which the deformation guide portion 131 is not embodied as a slot but is embodied as a membrane that is thinner than the side wall of the cap, if the syringe 1000 is strongly pressed down in the state of being inserted into the cap, the thin membrane is torn, and consequently, the upper end portion of the cap 130 is spread.

Since the situation after this is the same as described above, an explanation thereof will be omitted to avoid duplication.

Hereinafter, the operation of the second embodiment of the present invention will be described with reference to the attached drawings.

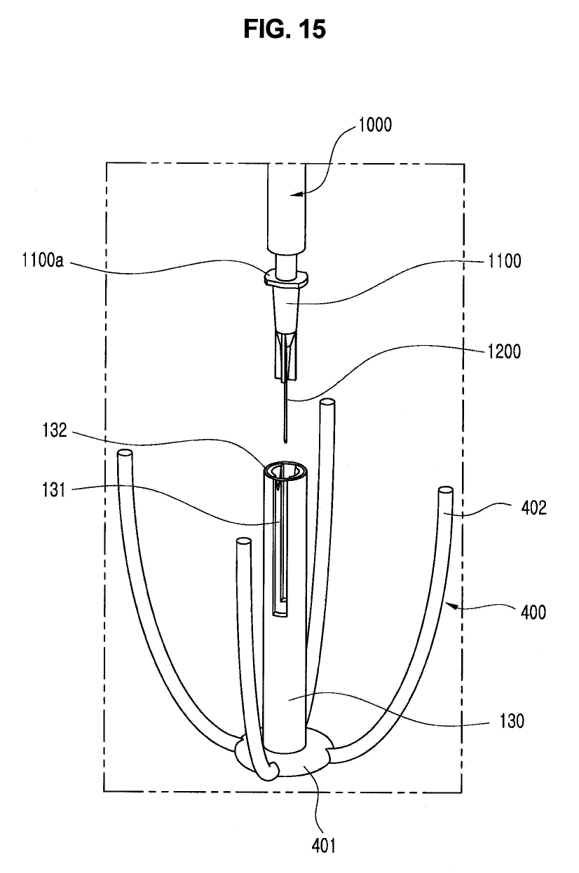

As illustrated in FIGS. 14 and 15, when a user places the safety cap assembly 1b according to the second embodiment of the present invention on a horizontal surface, the safety cap assembly stands upright and the insertion hole in the cap 130 is directed upwards due to the support part 400.

In this state, the user holds the syringe 1000, to which the needle 1200 and the needle-mounting part 1100 are coupled, with one hand, and inserts the needle 1200 and the needle-mounting part 1100 into the insertion hole in the cap 130.

When the user inserts the needle 1200 and the needle-mounting part 1100 into the cap 130, as illustrated in FIG. 12, the needle 1200 and the needle-mounting part 1100 can move until the stopping protrusion 1100a formed at the upper end of the needle-mounting part 1100 contacts the upper end of the cap 130.

Since the needle 1200 and the needle-mounting part 1100 are capable of being withdrawn back from the cap, it is possible to reuse the same.

In this state, the stopping protrusion 1100a of the needle-mounting part 1100 is in contact with the upper end of the cap 130 and the upper ends of the stopping parts 134. Therefore, the needle-mounting part 1100 is prevented from moving downwards.

That is, since the connection member 132 is maintained in a continuous shape and the deformation guide slot 131 is maintained in a non-spread shape, the stopping parts are not spread, thereby preventing the needle-mounting part 1100 from moving downwards.

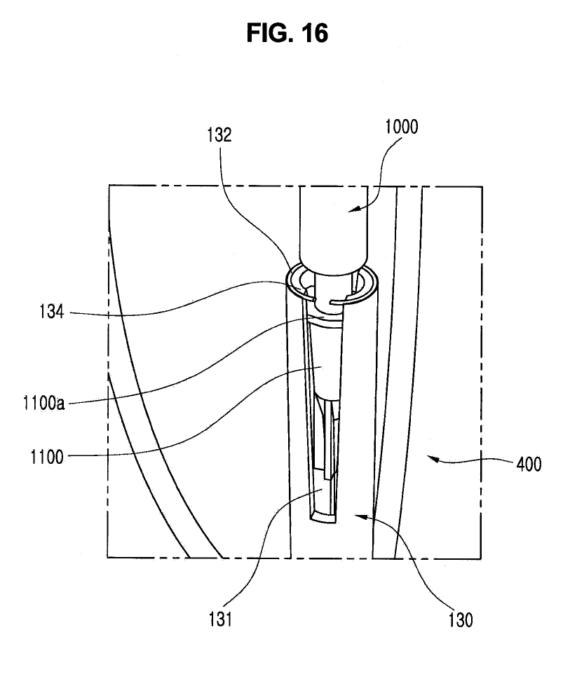

In this state, if the user strongly presses the syringe 1000 down in order to prevent reuse of the needle 1200, as illustrated in FIG. 16, the pressure acts as a force that spreads the upper end portion of the cap 1, and the connection member 132 does not endure such a force, and breaks.

Alternatively, in the case in which the deformation guide portion 131 is not embodied as a slot but is embodied as a membrane that is thinner than the side wall of the cap, if the syringe 1000 is strongly pressed down in the state of being inserted into the cap, the thin membrane is torn, and a crack is created in the torn portion of the membrane.

At the same time, the width of the deformation guide portion 131 is sharply increased, and the upper end portion of the cap 130 is spread widely.

Accordingly, the distance between the stopping parts 134 is also sharply increased. The needle-mounting part 1100 is easily inserted between the stopping parts, and the stopping protrusion 1100a of the needle-mounting part 1100 is located below the lower ends of the stopping parts 134.

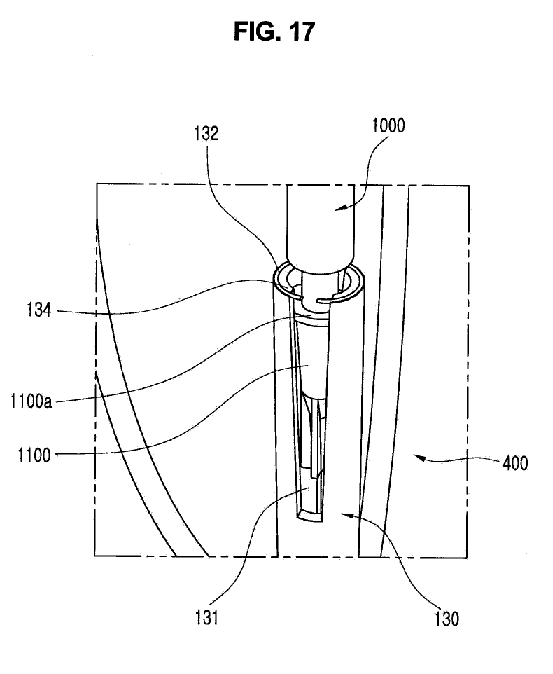

Thereafter, the upper end portion of the cap 130 is elastically restored and the insertion hole thereof retracts back, and, as illustrated in FIG. 17, the lower ends of the stopping parts 134 are placed on the upper end of the stopping protrusion 1100a of the needle-mounting part 1100.

Therefore, even if the user tries to pull the syringe 1000 up, the needle 1200 and the needle-mounting part 1100 are prevented from moving upwards and cannot be reused.

Hereinafter, the third embodiment of the present invention will be described.

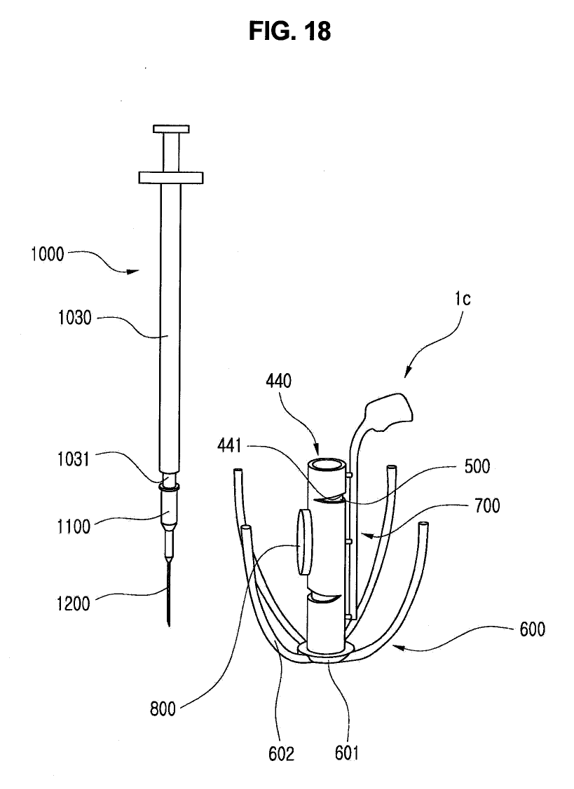

As illustrated in FIG. 18, a safety cap assembly 1c according to the third embodiment of the present invention comprises an accommodation cap 440, which has a given accommodation space formed therein, an inner cap 500, which is accommodated in the accommodation cap 440, a support part 600, which is connected to the lower end of the accommodation cap 440 and supports the accommodation cap 440 so that the accommodation cap 440 does not fall when placed on a horizontal surface, and a deformation prevention part 700, which is disposed beside the accommodation cap 440 along the longitudinal direction of the accommodation cap 440 so as to prevent deformation of the accommodation cap 440.

The inner cap 500 may be disposed in the accommodation cap 440, or alternatively, may be omitted.

That is, the present invention may have a dual-body structure including the accommodation cap 440 and the inner cap 500 or may have a single-body structure including only the accommodation cap 440.

A needle 1200, a needle-mounting part 1100, and a head portion 1031 of a main body 1030 of the syringe 1000, to which the needle-mounting part 1100 is coupled, may be inserted into the accommodation cap (or the inner cap).

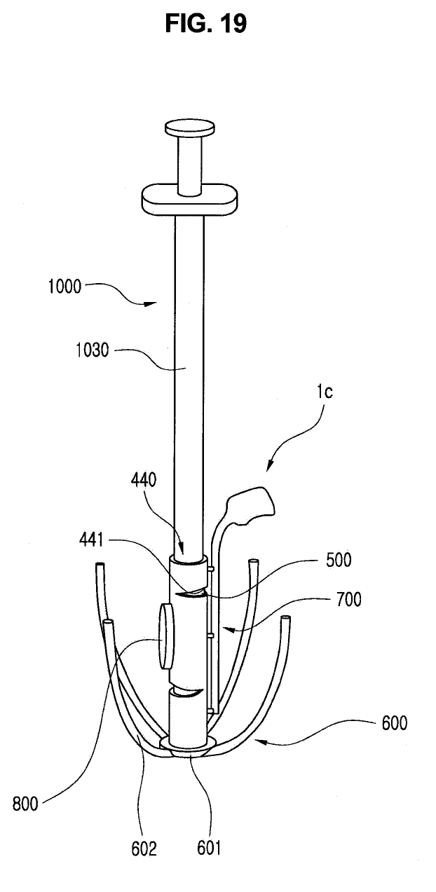

FIG. 19 is a view illustrating the state in which the needle 1200, the needle-mounting part 1100 and a part of the main body 1030 of the syringe are inserted into the accommodation cap 440 and are accommodated therein, and FIG. 18 is a view illustrating the state in which the entirety of the syringe 1000 is separated from the accommodation cap 440.

The accommodation cap 440 may be formed to have a cylindrical shape; however, the embodiment is not limited thereto, and it may have other shapes.

The accommodation cap 440 may have a deformation guide portion 441 formed in the side wall thereof, and the deformation guide portion 441 may be embodied as a slot, which widens from the interior of the accommodation cap to the exterior of the accommodation cap; however, the embodiment is not limited thereto.

It is preferable for the deformation guide portion 441 to be provided in a plural number and for the plurality of deformation guide portions 441 to be spaced apart from each other.

The plurality of deformation guide portions 441 may be preferably formed so as to be spread in different directions from each other. This is for the purpose of enabling the accommodation cap 440 to be bent in different directions.

The exterior and the interior of the accommodation cap 440 may communicate with each other due to the deformation guide portions 441, whereby some parts of the inner cap 500 may be observed from outside the accommodation cap, or may be exposed to the outside.

Meanwhile, the support part 600 is disposed at one end (the lower end) of the accommodation cap 440.

The support part 600 serves to prevent the accommodation cap 440 from falling when placed on a horizontal surface.

The support part 600 is disposed at one end (the lower end) of the accommodation cap 440, and includes a support plate 601, which has a larger diameter than the accommodation cap 440, and a support leg 602, which extends outwards from the support plate 601.

The support leg 602 extends from the outer periphery of the support plate 601 in the outward direction and is provided in a plural number, and the plurality of the support legs 602 are spaced apart from each other.

Preferably, the support leg 602 first extends from the outer periphery of the support plate 601 in the lateral direction, and is then bent upwards (or toward the remaining end (the upper end) of the accommodation cap) in the shape of a curved line.

Preferably, the support leg 602 is formed of a material that is capable of being elastically deformed while having a certain rigidity.

In one example, plastic may be used as the material for the support leg.

That is, each of the support legs 602 has the shape of a curved line, and each pair of support legs 602, which are arranged diametrically opposite each other, have a U-shape.

Since the support legs 602 are bent upwards in the shape of a curved line, even when the accommodation cap 440 is pushed over, it stands upright with respect to a horizontal surface due to the support legs 602. That is, the accommodation cap does not fall but is maintained in the state of standing upright on a horizontal surface in the manner of a roly-poly toy.

Therefore, the inlet portion of the accommodation cap 440 may be open upwards all the time.

In the state in which the inlet portion of the accommodation cap 440 is open upwards, the user is capable of holding the syringe 1000 with one hand and inserting the needle 1200, the needle-mounting part 1100 and the head portion 1031 of the main body of the syringe into the accommodation cap 440 through the inlet portion of the accommodation cap 440.

That is, conventionally, a user holds a syringe with one hand and inserts a needle into a general syringe accommodation cap held in the other hand, which leads to the risk of getting pricked by the needle and of bacterial infection; however, the user is capable of inserting the needle 1200 into the accommodation cap 440 without the necessity of holding the accommodation cap 440 in the hand due to the support part 600.

Meanwhile, beside the accommodation cap 440 are provided a deformation prevention part 700, for preventing deformation of the accommodation cap, and a support-surface-providing part 800 for providing a support surface that is easily held or pushed by the user so as to more conveniently deform the accommodation cap 440 after the deformation prevention part 700 is removed from the accommodation cap 440.

Here, the number of the support-surface-providing part 800 may be one or more.

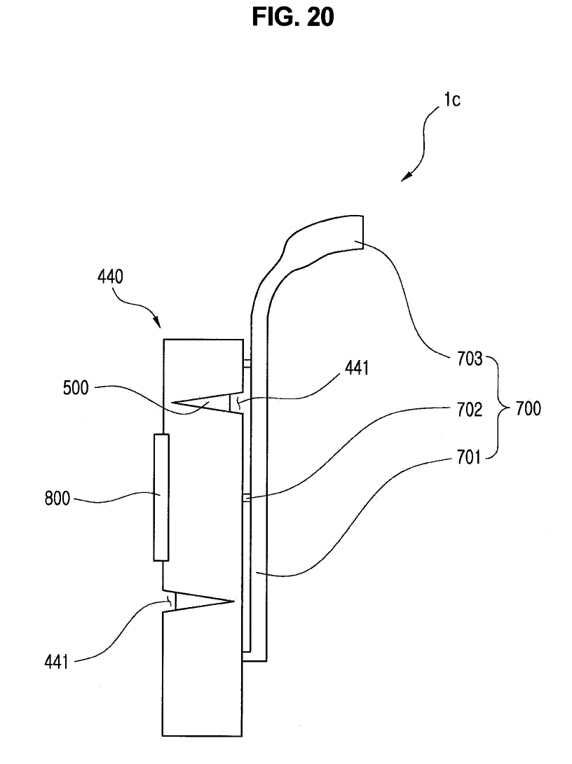

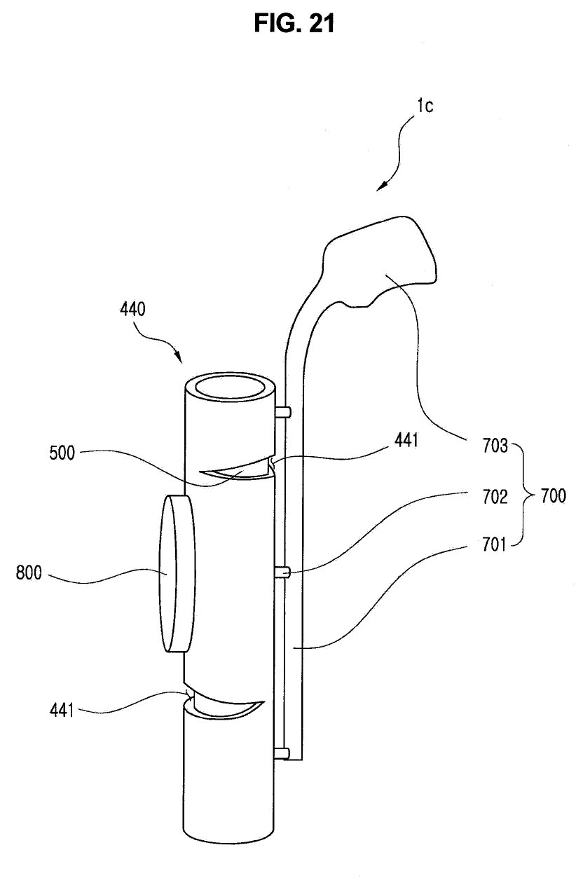

As illustrated in FIGS. 20 and 21, the deformation guide portion 441 formed at the accommodation cap 440 is formed to have a V-shape in the lateral direction.

When the accommodation cap 440 is deformed by the external force applied thereto, the accommodation cap 440 is bent about the deformation guide portion 441.

As described above, it is preferable for the plurality of deformation guide portions 441 to be disposed apart from each other at the upper portion and the lower portion of the accommodation cap and to be open in opposite directions. This is to serve the purpose of enabling the accommodation cap 440 to be bent several times.

However, there may be provided one deformation guide portion 441, in which case the accommodation cap 11 may be deformed once.

The deformation prevention part 700 is formed to have a shape resembling a walking cane, is disposed beside the accommodation cap along the longitudinal direction of the accommodation cap, and includes a support rod 701, which is disposed apart from the side surface of the accommodation cap, a connection portion 702, which connects the support rod 701 and the accommodation cap 440, and a handle portion 703, which extends from one end of the support rod 701 in a bent manner.

If the connection portion 702, which connects the support rod 701 and the accommodation cap 440, receives a predetermined force or larger, it may be cut off, may be separated from the accommodation cap 440, or may be separated from the support rod 701.

The connection portion 702 is provided in a plural number, and the plurality of connection portions 702 are spaced apart from each other in the longitudinal direction of the support rod 701 and the accommodation cap 440. Therefore, the support rod 701 may support the side surface of the accommodation cap 440 due to the connection portions 702.

Since the support rod 701 is disposed along the longitudinal direction of the accommodation cap 440 and reinforces the rigidity of the accommodation cap, the accommodation cap 440 is prevented from being deformed by bending.

The reason why the deformation prevention part 700 is needed is to prevent the needle or the main body of the syringe from being deformed by the unintentional bending of the accommodation cap 440 when the user uses the safety cap assembly 1c as a means for temporarily holding the syringe.

However, if the support rod 701 is removed from the accommodation cap 440, which will be described later, the aforementioned rigidity reinforcement effect disappears, which enables the user to easily bend and thus deform the accommodation cap 440.

The support-surface-providing part 800 is preferably provided with a support surface that is formed in a plate shape so as to enable the user to easily hold or push the same with his/her fingers, and particularly, has a concave portion formed in the center portion thereof so as to facilitate the user's holding or pushing manipulation.

Although the deformation prevention part 700 has been described as a structural element that is provided independently of the accommodation cap 440, the deformation prevention part 700 may not be formed as an independent structural element, but may be embodied as a reinforcement element that is disposed at the accommodation cap 440 so as to support the portions of the accommodation cap 440 that are expected to be bent.

Since the portions of the accommodation cap 440 that are expected to be bent are regions at which the deformation guide portions 441 are formed and surrounding regions around the deformation guide portions, which will be described later in detail, additional reinforcement elements (for example, pads or ribs) may be provided near the deformation prevention part 11 in the manner of being formed integrally with the accommodation cap 440 or of being attached to the accommodation cap 440, the reinforcement elements being capable of functioning as the deformation prevention part 700.

As illustrated in FIG. 22, if the user holds the handle portion 703 of the support rod 701 and pulls the support rod 701, the connection portions 702 may be cut off or separated from the accommodation cap 440, thereby releasing the connection between the support rod 701 and the accommodation cap 440.

In this state, if the user holds the accommodation cap 440 and bends the same, the accommodation cap 440 and the inner cap 500 may be easily bent about the deformation guide portions 441.

The accommodation cap 440 or the inner cap 500 may preferably be made of a material that is easily plastically deformed when a predetermined bending force or higher is applied thereto, for example plastic.

Hereinafter, the processes of keeping and discarding the syringe using the safety cap assembly 1c according to the third embodiment of the present invention will be described.

The safety cap assembly 1c is initially packaged in a package, and the support legs 602 are in the state of being retracted to the maximum extent and of being located near the accommodation cap 440.

When the user tears the package and takes the safety cap assembly 1c out of the package, the support legs 602 are spread, as illustrated in FIG. 18.

In this state, when the user places the safety cap assembly 1c on a horizontal surface, the safety cap assembly 1c wobbles laterally and stands upright, and the inlet in the accommodation cap 440 is directed upwards.

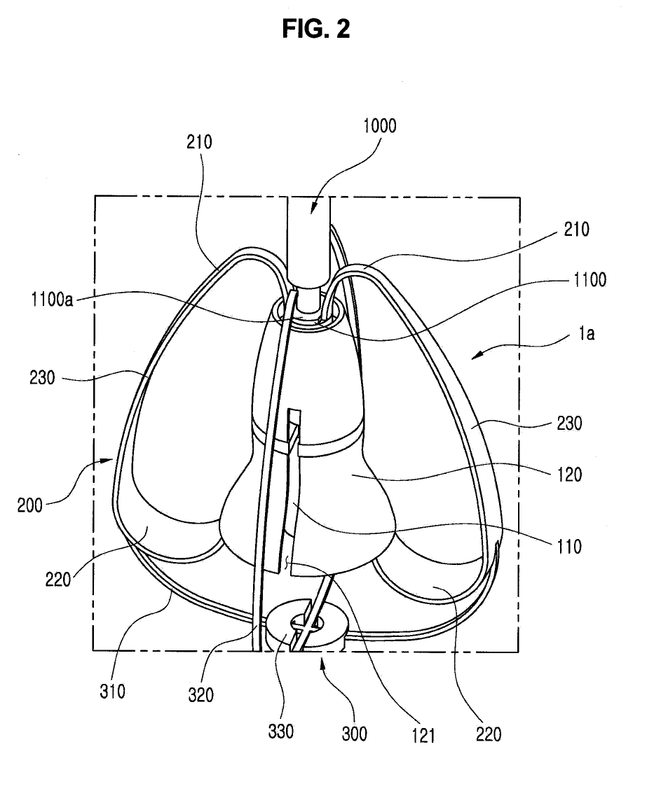

In this state, the user holds the main body 1030 of the syringe, which is to be discarded, and inserts the needle 1200, the needle-mounting part 1100, and the head portion 1031 of the main body of the syringe into the accommodation cap (the inner cap) 10 and 20, which is illustrated in FIG. 2.

In this state, the user holds the handle portion 703 of the deformation prevention part 700 and tears the support rod 701 of the deformation prevention part 700 off the accommodation cap 440.

In the state in which the deformation prevention part 700 is removed from the accommodation cap 440, when the user holds the accommodation cap 440 and bends the same about the deformation guide portion 441, as illustrated in FIG. 6, the deformation prevention part 11 may be widened, and the accommodation cap 440 and the inner cap 500 may be deformed into an L-shape, whereby the needle 1200 may also be deformed into an L-shape.

When the user bends the deformed portions further, the accommodation cap and the inner cap may be deformed into a U-shape, and consequently, the needle 1200 may also be deformed into a U-shape, as illustrated in FIG. 7.

At this time, if the user puts his/her finger to the support-surface-providing part 800 and applies force thereto, the deformation may be easily realized.

And, as illustrated in FIG. 8, when the user deforms the accommodation cap 440 about another deformation prevention slot 11, pressure is applied to the head portion 1031 of the main body of the syringe, which is connected to the needle-mounting part 1100, and the head portion 11 of the main body of the syringe is broken, whereby the needle-mounting part 1100 is separated from the remaining part of the main body 1030 of the syringe.

In order to clearly show the deformed or broken state of the needle 1200 or the head portion 1031 of the main body of the syringe when the accommodation cap 440 is deformed, the needle 1200, the needle-mounting part 1100 and the main body 1030 of the syringe are illustrated as being located outside the accommodation cap 440 in FIGS. 6 to 8.

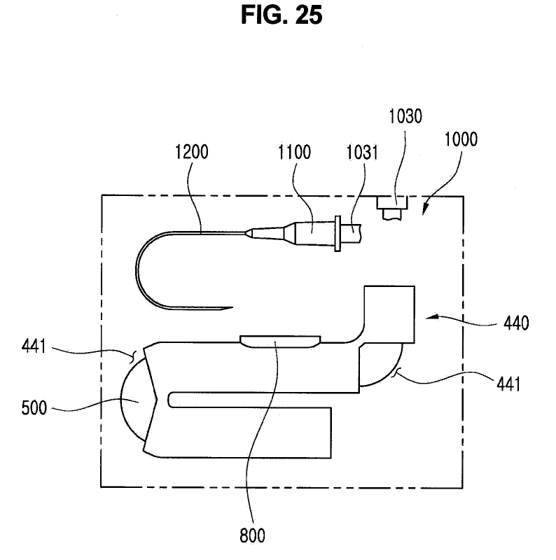

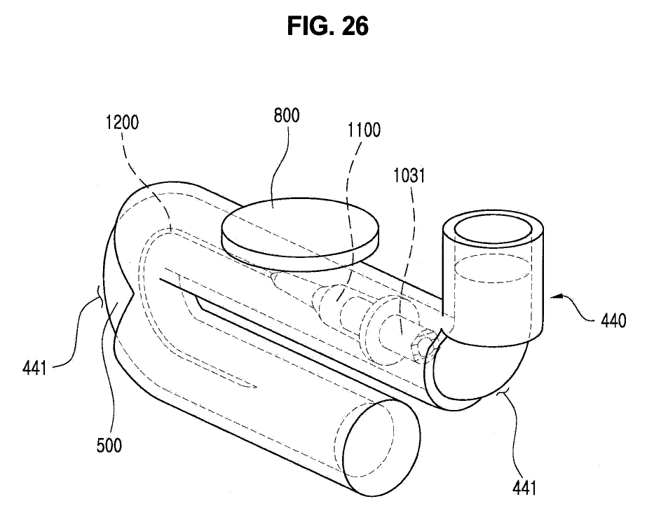

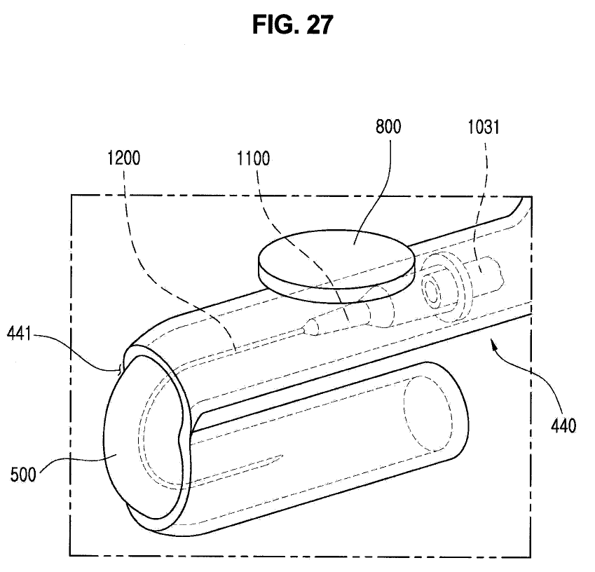

When the needle 1200, the needle-mounting part 1100 and the head portion 1031 of the main body of the syringe are finally deformed into the state illustrated in FIG. 25 while accommodated in the accommodation cap 440 and the inner cap 500, the needle 1200, the needle-mounting part 1100 and the head portion 1031 of the main body of the syringe are in the state of being locked in the accommodation cap 440 and the inner cap 500, as illustrated in FIGS. 26 and 27.

Since the lower end portion of the accommodation cap 440 is blocked and the upper end portion of the accommodation cap 440 is bent by the deformation guide portion 401 so as to interrupt the broken portion of the head portion 1031 of the main body of the syringe, the needle 1200, the needle-mounting part 1100 and the broken portion of the head portion 1031 of the main body of the syringe are prevented from escaping outside the accommodation cap 440.

That is, when the accommodation cap 440 is deformed into an L-shape or a U-shape about the deformation guide portions 441, the bent portions thereof serve as an escape prevention structure for preventing the needle 1200, the needle-mounting part 1100 and the broken portion of the head portion 1031 of the main body of the syringe from escaping outside the accommodation cap 440.

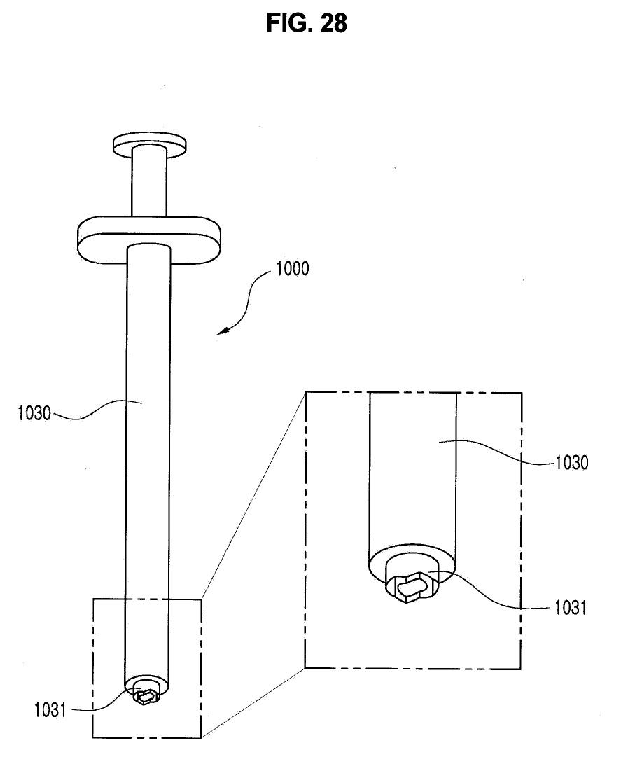

Since the main body 1030 of the syringe is damaged as illustrated in FIG. 28 when the head portion 1031 of the main body of the syringe is broken, the syringe must inevitably be discarded, and it is absolutely impossible to reuse the syringe by coupling the needle-mounting part 1100 to the damaged portion.

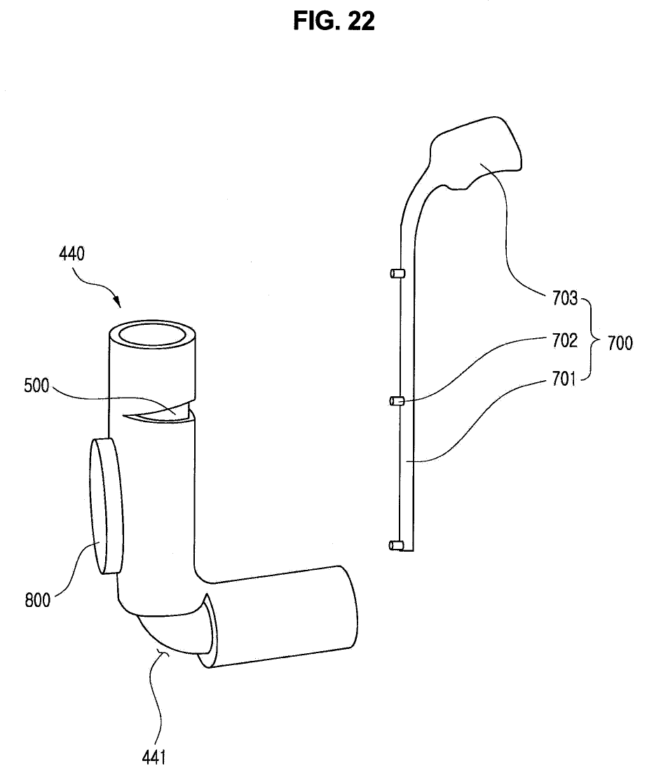

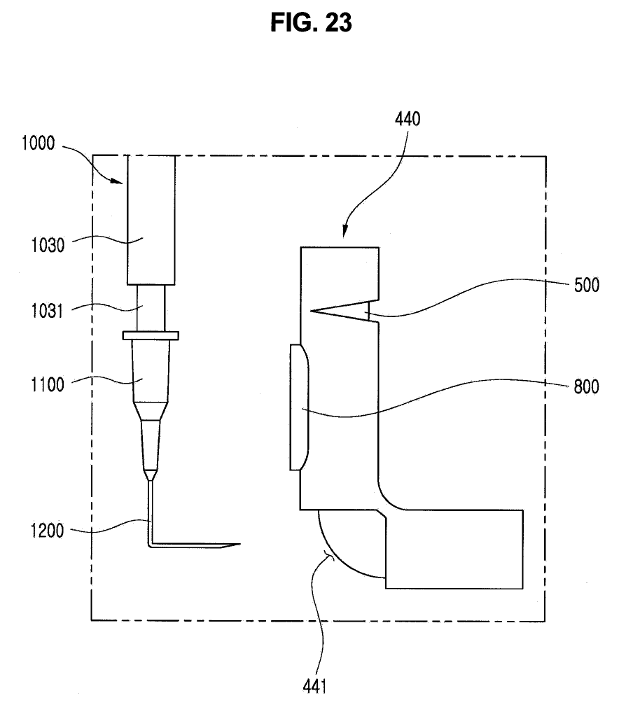

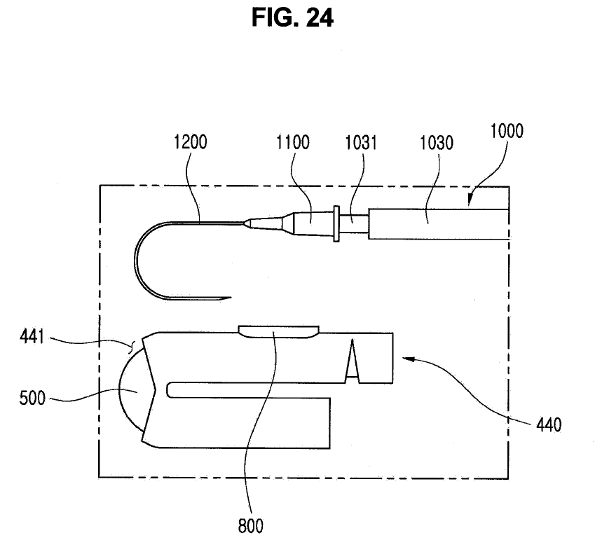

Moreover, the needle 1200, the needle-mounting part 1100 and the head portion 1031 of the main body of the syringe, which are deformed or damaged through the processes described with reference to FIGS. 23 to 27, are locked in the accommodation cap 440, whereby it becomes absolutely impossible to reuse them.

Particularly, if the user spreads the portion of the accommodation cap at which the needle 1200 is located, the needle 1200 may even break, which makes it further impossible to reuse the syringe.

Therefore, after the user throws away the safety cap assembly 1c and the elements of the syringe locked therein, having been subjected to the processes described above with reference to FIGS. 23 to 27, even if someone intends to reuse them, whether maliciously or mistakenly, the reuse thereof is completely prevented.

The syringe, which is configured to be inserted into the syringe safety caps 1a, 1b and 1c according to the first to third embodiments described above, is also a subject matter of the present invention.

The syringe 1000 according to the present invention functions to filter foreign substances, such as fine glass splinters, from liquid medication and thus to prevent such foreign substances from being injected into a human body.

Hereinafter, the syringe 1000 having such a function will be explained in detail.

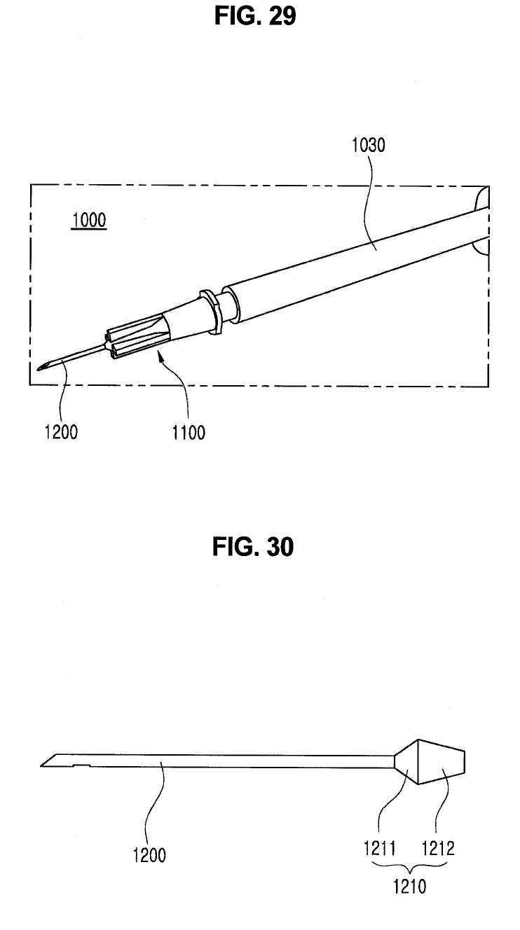

As illustrated in FIG. 29, the syringe 1000 according to the present invention comprises a syringe cylinder 1030, in which a liquid such as a medication is temporarily retained, a needle-mounting part 1100, which has the configuration of a support cap coupled to a nozzle provided at the front end of the syringe cylinder 1030, and a needle 1200, which is fitted into the needle-mounting part 1100.

The rear end portion of the needle 1200 is secured to the front end portion of the needle-mounting part 1100.

A syringe plunger (not illustrated) is provided in the syringe cylinder 1030.

When the syringe plunger moves from the front portion to the rear portion of the internal space in the syringe cylinder 1030, the liquid is introduced into the syringe cylinder 1030 via the needle 1200 and the needle-mounting part 1100.

Conversely, when the syringe plunger moves from the rear portion to the front portion of the internal space in the syringe cylinder 1030, the liquid is discharged outside the syringe cylinder 1030 via the needle-mounting part 1100 and the needle 1200.

FIGS. 30 to 33 are views illustrating all constitutional components provided in the needle-mounting part 1100.

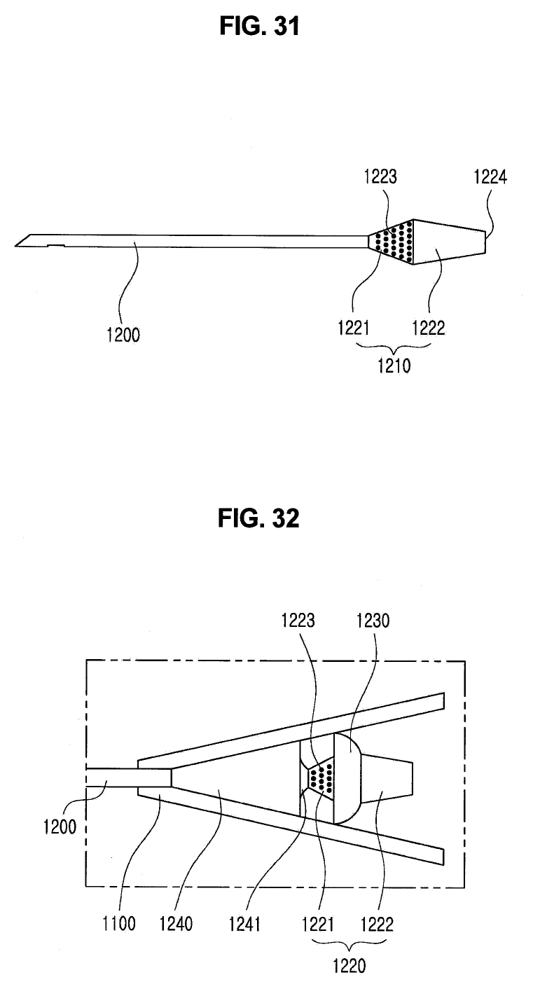

FIG. 30 is a view illustrating a filter member 1210, and FIG. 31 is a view illustrating a filter cover 1220.

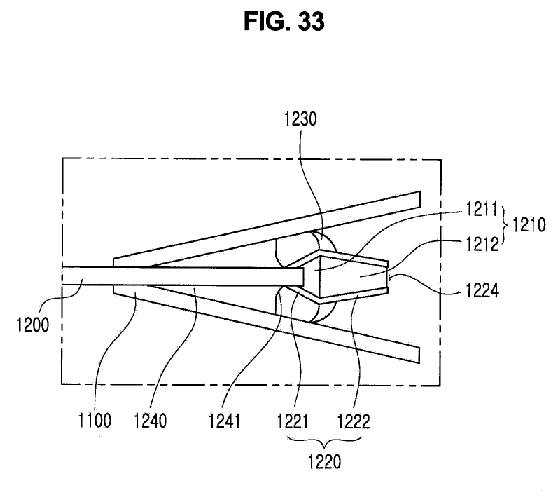

FIGS. 32 and 33 are views illustrating a valve member 1230, which is disposed near the filter cover 1220, and a guide member 1240, which is disposed in front of the filter cover 1220.

Inside the needle-mounting part 1100 are provided the rear end portion of the needle 1200, the filter member 1210 coupled to the rear end portion of the needle 1200, the filter cover 1220, the valve member 1230, and the guide member 1240.

The filter member 1210 has an internal structure having a high density and is formed of a fiber material or some other soft material. The role of the filter member 1210 is to filter foreign substances from the liquid medication when they are introduced into the needle-mounting part.

The filter member 1210 does not have a cylindrical or polyhedral shape, but instead has a shape such that the diameter thereof is gradually increased and then gradually decreased.

The region in which the diameter is increased from front to back will be referred to as a first filter region 1211, and the region in which the diameter is decreased will be referred to as a second filter region 1212.

As illustrated in FIGS. 31 and 33, the filter cover 1220 is provided outside the filter member 1210 in order to support the filter member 1210.

The filter cover 1220 preferably has a shape corresponding to the shape of the filter member 1210.

Here, the filter cover 1220 is divided into a first cover region 1221 and a second cover region 1222, the first filter region 1211 is accommodated in the first cover region 1221, and the second filter region 1212 is accommodated in the second cover region 1222.

That is, the first cover region 1221 is the region in which the diameter of the filter cover 1220 is increased from front to back, and the second cover region 1222 is the region in which the diameter of the filter cover 1220 is decreased from back to front.

The first cover region 1221 has a plurality of through-holes 1223 formed in the outer peripheral surface thereof, and the second cover region 1222 has an opening 1224 formed in the rear end portion thereof.

When the liquid is discharged outside, it flows via the through-holes 1223, and when the liquid is introduced into the syringe cylinder 1030, it flows via the opening 1224.

As illustrated in FIGS. 32 and 33, the valve member 1230 is provided around the outer peripheral surface of the filter cover 1220.

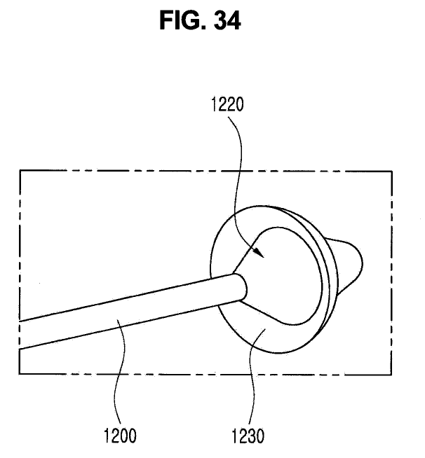

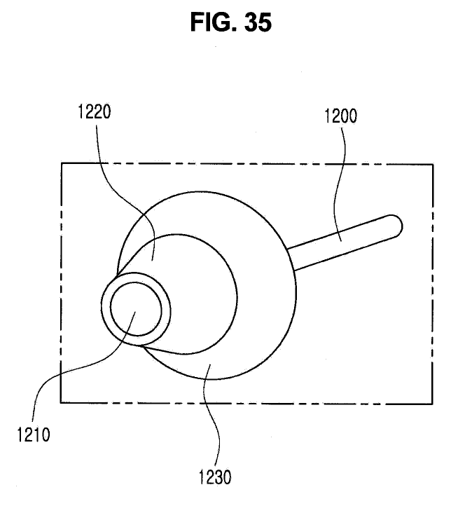

FIGS. 34 and 35 show a front perspective view and a rear perspective view of the valve member 1230, respectively.

The valve member 1230 may have the configuration of a diaphragm that extends from the outer peripheral surface of the filter cover 1220 in the radial direction and selectively contacts or moves away from the inner peripheral surface of the needle-mounting part 1100.

Preferably, the valve member 1230 may be mounted adjacent to the boundary between the first filter region 1221 and the second filter region 1222.

The valve member 1230 may have a shape such that the thickness thereof is decreased from the center to the outer periphery thereof; however, the thickness thereof may be uniform.

On the other hand, the valve member 1230 may be preferably bent forwards from the connection portion with the filter cover 1220 in the radial direction, which serves the purpose of facilitating contact with or separation from the inner peripheral surface of the needle-mounting part 1200.

The front end portion of the needle-mounting part 1200 has a shape such that the inner diameter thereof is increased from front to back, whereby the outer periphery of the valve member 1240, which is bent forwards, may be easily brought into contact with or separated from the inner surface of the needle-mounting part 1200 depending on the operation of the syringe plunger and the liquid flow direction corresponding thereto.

On the other hand, as illustrated in FIGS. 32 and 33, the guide member 1240 is provided in the foremost portion of the internal space of the needle-mounting part 1200.

The guide member 1240 is disposed in front of the filter cover 1220. The guide member 1240 serves to change the flow direction of the liquid that has passed through the bypass passage formed by opening the valve member 1240 and to guide the liquid so that the liquid smoothly moves toward the through-holes 1223.

To this end, the guide member 1240 has a curved guide surface 1241 formed at the rear end thereof.

Therefore, the liquid, which has passed through the opened valve member 1230, is changed in flow direction by the guide member 1240, passes through the through-holes 1223, and moves to the needle 1200 via the first filter region 1211.

Hereinafter, the operation of the syringe 1000 according to the present invention will be described with reference to the attached drawings.

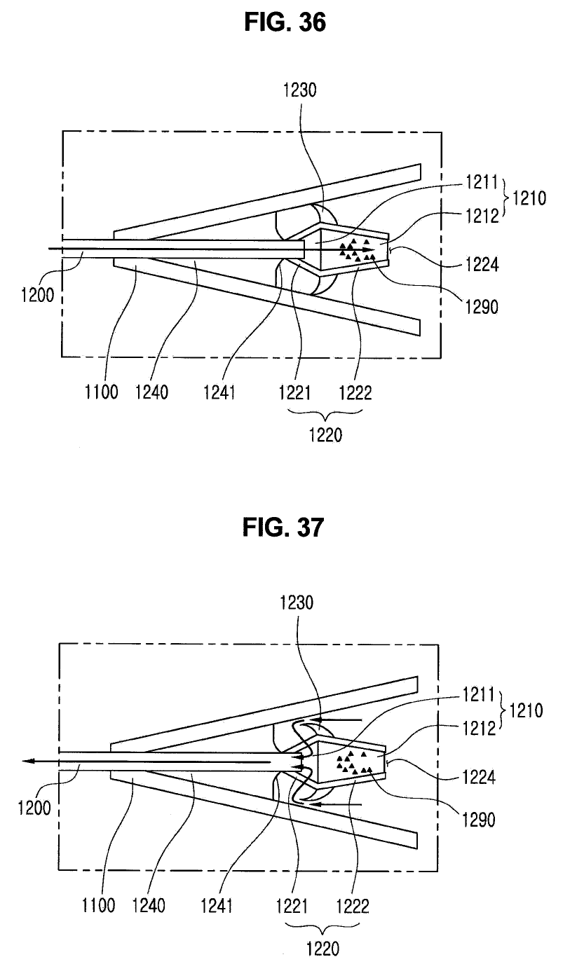

As illustrated in FIG. 36, when the user pulls the syringe plunger and a liquid is introduced into the syringe, a low-pressure region is created behind the valve member 1230 and a high-pressure region is created in front of the valve member 1230, whereby the outer periphery of the valve member 1230 is smoothly spread and is brought into contact with the inner peripheral surface of the needle-mounting part 1200.

Therefore, the space in front of the valve member 1230 and the space behind the valve member 1230 are segregated from each other, and the liquid, which has passed through the filter member 1210, is incapable of flowing out through the through-holes 1223. Instead, the liquid, which has passed through the filter member 1210, is capable of flowing out to the syringe cylinder through the opening 1224 in the filter cover 1220.

If foreign substances 1290 are present in the liquid, they may be filtered out by the filter member 1210, and most of them may remain in the second filter region 1212, which is the rear part of the filter member 1210.

Meanwhile, as illustrated in FIG. 37, when the user pushes the syringe plunger and the liquid is discharged, a high-pressure region is created behind the valve member 1230, and a low-pressure region is created in front of the valve member 1230, whereby the outer periphery of the valve member 1230 is pushed by the liquid, retracts back, and is separated from the inner peripheral surface of the needle-mounting part 1200.

Therefore, the space in front of the valve member 1230 and the space behind the valve member 1230 communicate with each other.

Due to the communication of the spaces with each other to thus have a larger area than the opening 1224 in the filter cover 1220, the resistance to the liquid flow through the filter cover 1220, and the difference in length of the flow paths in the filter member 1210, most of the liquid to be discharged passes through the bypass passage formed between the valve member 1230 and the inner wall of the needle-mounting part 1100 and is discharged outside through the needle 1200 via the through-holes 1223 and the first region 1211 of the filter member 1210.

When the liquid moves toward the through-holes 1223, it is changed in flow direction and is guided by the guide surface 1241 of the guide member 1240.

On the other hand, when the foreign substances 1290 are present in the liquid that is introduced into the syringe, most of the foreign substances 1290 remain in the second region 1212 of the filter member 1210; however, since most of the liquid to be discharged flows through the first filter region 211 and the liquid flowing through the first filter region 1211 prevents the foreign substances from moving toward the needle, the foreign substances may be prevented from escaping and moving toward the needle.

The reason for this is that the liquid flowing through the first filter region 1211 applies a force to the foreign substances 1290 in the direction opposite the needle 1200.

Meanwhile, during the discharge operation, the liquid may also move toward the filter member 1210 through the opening 1224; however, as described above, the amount of liquid flowing toward the opening 1224 is much smaller than the amount of liquid flowing toward the through-holes 1223 because the filter member 1210 has a high density, the opening 1224 has a smaller area than the bypass passage (the passage formed by the operation of opening the valve member), and the liquid, which is directed toward the needle 1200 via the opening 1224, must flow through both the first filter region 1211 and the second filter region 1212, whereas the liquid having passed through the bypass passage flows only through the first filter region 1211.

Therefore, according to the above-described present invention, even though a liquid mixed with fine glass splinters of a glass ampoule is introduced into the syringe, such foreign substances are filtered out during the liquid introduction process, and the foreign substances are prevented from being discharged during the liquid discharge process by providing a liquid discharge passage separately from a liquid introduction passage and enabling the liquid flowing through the discharge passage to apply force to the foreign substances in the direction opposite the discharge direction.

Although the present invention has been described with reference to embodiments illustrated in the drawings, it is to be understood that the foregoing embodiments are merely exemplary and various modifications or equivalent embodiments thereof may be made from the detailed description of the present invention by those skilled in the art.

Accordingly, the true scope of the present invention should be determined by the technical spirit of the claims.

1. A safety cap assembly for a syringe, comprising:
an inner cap for receiving a needle and a needle-mounting part of the syringe inserted thereinto;
an outer cap for accommodating the inner cap therein, the outer cap having a deformation guide slot formed in a side surface thereof so as to be spread by a predetermined pressure applied thereto;
a connection part for connecting the inner cap and the outer cap, the connection part being configured so as to be elastically deformed; and
a support part connected to at least one of the connection part and the outer cap, the support part supporting and maintaining the inner cap, the outer cap and the connection part spaced apart from a horizontal surface and providing a support force to prevent the inner cap, the outer cap and the connection part from falling down.

2. The safety cap assembly according to claim 1, wherein the connection part includes:
a first connection portion connected to an upper end portion of the inner cap;
a second connection portion connected to a lower end portion or a side surface of the outer cap; and
a third connection portion for connecting the first connection portion and the second connection portion, the third connection portion being disposed outside the outer cap,
and wherein the connection part has a lower end located below a lower end of the outer cap.

3. The safety cap assembly according to claim 1, further comprising:
a locking part provided between the inner cap and the outer cap so as to maintain a position of the inner cap; and
a locking recess formed in an outer peripheral surface of the inner cap so as to mount the locking part to the inner cap.

4. The safety cap assembly according to claim 3, wherein the locking part is provided in a plural number, and the plurality of locking parts is inserted into the outer cap such that the locking parts protrude inwards from an inner surface of the outer cap so as to be fitted into the locking recess in the inner cap.

5. The safety cap assembly according to claim 1, further comprising:
a support ring mounted to an inner peripheral surface of the outer cap in order to surround an outer peripheral surface of the inner cap,
wherein the support ring has a sectional shape such that an inner side surface thereof extends downwards and is slanted inwards so that, when the needle-mounting part fitted into the inner cap moves downwards and is then located below the support ring, a periphery of the support ring is located on an upper end of the needle-mounting part, thereby preventing the needle-mounting part from moving upwards.

6. The safety cap assembly according to claim 1, wherein the support part includes:
a support plate configured to be placed on a horizontal surface;
a first support portion passing through the support plate in a first direction and having two ends bent upwards in a shape of a curved line and connected to the connection part; and
a second support portion passing through the support plate in a second direction so as to cross the first support portion and having two ends bent upwards in a shape of a curved line,
wherein the first support portion and the second support portion are made of an elastically deformable material and provide the support force together with the support plate.

7. A safety cap assembly for a syringe, comprising:
a cap for receiving a needle and a needle-mounting part of the syringe inserted thereinto, the cap having a deformation guide portion formed in a side surface thereof so as to be spread by a predetermined pressure applied thereto;
a stopping part mounted to an inner peripheral surface of the cap so as to surround an outer peripheral surface of the needle-mounting part inserted into the cap; and
a connection member provided at the cap or the stopping part so as to prevent deformation of the deformation guide portion when the pressure applied to the cap is lower than a predetermined value, the connection member being configured to be broken when the pressure applied to the cap has the predetermined value or higher and thus the deformation guide portion is spread.

8. The safety cap assembly according to claim 7, wherein, when the needle-mounting part moves downwards and an upper end of the needle-mounting part is located below the stopping part, a periphery of the stopping part is located on an upper end of the needle-mounting part, thereby preventing the needle-mounting part from moving upwards, and
the stopping part has a sectional shape such that an inner side surface thereof extends downwards and is slanted inwards.

9. The safety cap assembly according to claim 7, further comprising:
a stopping part mounted to an inner peripheral surface of the cap so as to surround an outer peripheral surface of the needle-mounting part inserted into the cap,
wherein the stopping part is provided in a plural number and the plurality of stopping parts are spaced apart from each other, and when the needle-mounting part passes through the stopping parts, the stopping parts are temporarily spread away from each other and then retract back to cover a stopping protrusion of the needle-mounting part, thereby preventing the needle-mounting part from escaping the cap in a direction opposite an insertion direction.

10. The safety cap assembly according to claim 7, further comprising:
a shape-maintaining member provided at the cap or the stopping part, the shape-maintaining member being configured to maintain a shape of the cap when the syringe is inserted into the cap with a pressure lower than a predetermined value.

11. A safety cap assembly for a syringe, comprising:
an accommodation cap having a space formed therein to accommodate a needle and at least a part of a main body of the syringe inserted thereinto, the accommodation cap being configured so as to be deformed by an external force applied thereto, thereby causing deformation of the needle or the main body of the syringe accommodated therein; and
a support part connected to the accommodation cap, the support part providing a support force so as to prevent the accommodation cap placed on a horizontal surface from falling down,
wherein the support part has an overall shape of a curved line, and guides the accommodation cap placed on the horizontal surface so that the accommodation cap wobbles laterally and finally stands upright with respect to the horizontal surface.

12. The safety cap assembly according to claim 11, further comprising:
a deformation guide portion for enabling the accommodation cap to be easily deformed when an external force is applied to the accommodation cap.

13. The safety cap assembly according to claim 12, wherein, when a user applies an external force of a predetermined value or higher to the accommodation cap in order to deform the accommodation cap, the deformation guide portion is enlarged and the accommodation cap is bent, thereby causing permanent deformation or cutting of the needle or the main body of the syringe accommodated in the accommodation cap.

14. The safety cap assembly according to claim 11, further comprising:
a deformation prevention part provided outside the accommodation cap or provided near a deformation guide portion of the accommodation cap in a manner of being formed integrally with the accommodation cap or of being embodied as an additional reinforcement structure, the deformation prevention part being configured to prevent the accommodation cap from being bent.

15. The safety cap assembly according to claim 11, further comprising:
a support-surface-providing part disposed on an outer surface of the accommodation cap, the support-surface-providing part providing a support surface for enabling a user to easily hold or push the accommodation cap when applying an external force to the accommodation cap.

16. The safety cap assembly according to claim 11, wherein the support part includes a plurality of support legs spaced apart from each other, the support legs being connected to a lower end portion of the accommodation cap and bent upwards in a shape of a curved line.

17. The safety cap assembly according to claim 11, wherein, when the accommodation cap is deformed while the needle or at least a part of the main body of the syringe is accommodated in the accommodation cap, the accommodation cap forms an escape prevention structure for preventing the deformed needle or a deformed or broken portion of the main body of the syringe from escaping outside the accommodation cap.

18. The safety cap assembly according to claim 17, wherein the escape prevention structure is realized by a bent portion of the accommodation cap, and the deformed needle or the deformed or broken portion of the main body of the syringe is caught in the bent portion of the accommodation cap.

19. The safety cap assembly according to claim 17, further comprising:
a deformation guide portion for enabling the accommodation cap to be easily deformed when an external force of a predetermine value or higher is applied to the accommodation cap,
wherein the escape prevention structure is realized by enlargement of the deformation guide portion.

20. The safety cap assembly according to claim 17, further comprising:
a deformation prevention part provided outside the accommodation cap or provided near a deformation guide portion of the accommodation cap in a manner of being formed integrally with the accommodation cap or of being embodied as an additional reinforcement structure, the deformation prevention part being configured to prevent the accommodation cap from being bent; and
a support-surface-providing part disposed on an outer surface of the accommodation cap, the support-surface-providing part providing a support surface for enabling a user to easily hold or push the accommodation cap when applying an external force to the accommodation cap.

21. A syringe comprising:
a needle-mounting part for supporting a needle;
a filter member provided in the needle-mounting part and mounted to an end of the needle;
a filter cover disposed outside the fitter member and having a through-hole formed in an outer peripheral surface thereof and an opening formed in an end portion thereof; and
a valve member disposed on an outer peripheral surface of the filter cover,
wherein, when a liquid is introduced into the syringe by an introduction operation of a plunger, the valve member segregates a space in the needle-mounting part so as to induce introduction of the liquid toward the opening, and when the liquid is discharged by a discharge operation of the plunger, the valve member releases the segregation of the space in the needle-mounting part so as to induce discharge of the liquid via the through-hole and forms a passage for discharge of the liquid, the passage for discharge of the liquid being different from a passage for introduction of the liquid.

22. The syringe according to claim 21, wherein the valve member has a configuration of a diaphragm,
during the introduction operation of the plunger, the valve member is operated such that an outer periphery thereof is brought into contact with an inner peripheral surface of the needle-mounting part, thereby segregating a space in front of the valve member and a space behind the valve member from each other, and
during the discharge operation of the plunger, the valve member is operated such that the outer periphery thereof is separated from the inner peripheral surface of the needle-mounting part, thereby enabling the space in front of the valve member and the space behind the valve member to communicate with each other.

23. The syringe according to claim 22, wherein the needle-mounting part has a front end portion having a shape such that an inner diameter thereof is increased from front to back, and
the valve member extends from an outer peripheral surface of the filter cover in a radial direction and is bent forwards so that, when the liquid moves from back to front, the valve member is separated from the inner peripheral surface of the needle-mounting part, and when the liquid moves from front to back, the valve member is brought into contact with the inner peripheral surface of the needle-mounting part.

24. The syringe according to claim 21, further comprising:
a guide member disposed in a foremost portion of an internal space of the needle-mounting part, the guide member being configured to guide the liquid having passed through the valve member toward the through-hole during the discharge operation of the plunger.

25. The syringe according to claim 24, further comprising:
a guide surface having a curved shape and provided at the guide member so as to guide the liquid toward the through-hole.

26. The syringe according to claim 21, wherein the filter cover includes:
a first cover region formed such that a diameter thereof is increased from front to back; and
a second cover region formed such that a diameter thereof is decreased from front to back,
wherein the through-hole is provided in a plural number and the plurality of through-holes is formed in an outer peripheral surface of the first cover region, and the opening is formed in a rear end of the second cover region.

27. The syringe according to claim 26, wherein the valve member is disposed adjacent to a boundary between an outer peripheral surface of the first cover region and an outer peripheral surface of the second cover region.

28. The syringe according to claim 21, wherein, when foreign substances are mixed with the liquid to be introduced into the syringe and the liquid mixed with the foreign substances is introduced toward a cylinder of the syringe by the introduction operation of the plunger, the foreign substances are filtered out by the filter member, the liquid moves toward the opening and is introduced into the cylinder, and the valve member performs a blocking operation, thereby preventing introduction of the liquid via the through-hole.

29. The syringe according to claim 28, wherein, when the liquid is discharged from the cylinder by the discharge operation of the plunger, the valve member performs an opening operation so that the liquid in the cylinder passes between the valve member and an inner wall of the needle-mounting part and is discharged outside through the needle via the through-hole.

30. The syringe according to claim 29, wherein the liquid discharged via the through-hole applies a force to the foreign substances remaining in the filter member in a direction opposite the needle.
